(19) United States
(12) Patent Application Publication
(10) Pub. No.: US 2015/0025329 A1
(43) Pub. Date: Jan. 22, 2015

(54) PATIENT CARE SURVEILLANCE SYSTEM AND METHOD

(71) Applicant: Parkland Center for Clinical Innovation, Dallas, TX (US)

(72) Inventors: Rubendran Amarasingham, Dallas, TX (US); Vaidyanatha Siva, Plano, TX (US); Monal Shah, Dallas, TX (US); Anand Shah, Dallas, TX (US); George Oliver, Southlake, TX (US); Praseetha Cherian, Sunnyvale, TX (US); Javier Velazquez, Lewisville, TX (US); Paul Mayer, III, Dallas, TX (US)

(21) Appl. No.: 14/326,863

(22) Filed: Jul. 9, 2014

Related U.S. Application Data
(60) Provisional application No. 61/847,852, filed on Jul. 18, 2013.

(51) Int. Cl. A61B 5/00; A61B 5/0205; A61B 5/145

(52) U.S. Cl. CPC A61B 5/0022; A61B 5/14532; A61B 5/0205; A61B 5/7282; A61B 5/7275; A61B 5/742; A61B 5/7405
USPC 600/301; 600/300; 600/365

(57) ABSTRACT

A patient care surveillance system comprises a data store operable to receive and store clinical and non-clinical data associated with at least one patient, a user interface configured to receive user input of current information related to at least one patient, a monitor configured to sense at least one parameter associated with at least one patient, and further configured to generate real-time patient monitor data, a data analysis module configured to access the data store and analyze the clinical and non-clinical data, receive and analyze the current information and real-time patient monitor data, and identify at least one adverse event associated with the care of at least one patient, and a data presentation module operable to present information associated with at least one adverse event to a healthcare professional, the information including contextual information associated with the adverse event.

### RELATED APPLICATION

This application claims the benefit of U.S. Provisional Patent Application Ser. No. 61/847,852, entitled “Patient Care Surveillance System and Method,” and filed on Jul. 18, 2013.

### FIELD

The present disclosure generally relates to a healthcare system, and more particularly it relates to a patient care surveillance system and method.

### BACKGROUND

Hospitals and other healthcare facilities have been attempting to monitor and quantify the occurrence of adverse events within the facilities to improve the quality of patient care. An adverse event is typically defined as unintended injury to a patient resulting from or contributing to medical care that requires additional monitoring, treatment, or hospitalization, or that results in death. Conventionally, hospitals and healthcare facilities rely on voluntary incident reporting and retrospective manual record reviews to identify and track adverse events. These past efforts have been largely unreliable, fail to capture all relevant data and do not present an accurate and timely picture of patient care. In addition, because of their voluntary nature, many adverse events are never reported.

### BRIEF DESCRIPTION OF THE DRAWINGS

FIG. 1 is a simplified block diagram of an exemplary embodiment of a patient care surveillance system and method according to the present disclosure;

FIG. 2 is a simplified block diagram of exemplary information input and output of a patient care surveillance system and method according to the present disclosure;

FIG. 3 is a simplified flowchart of an exemplary embodiment of a patient care surveillance system and method according to the present disclosure; and

FIGS. 4-25 are exemplary screen displays of a patient care surveillance system and method according to the present disclosure.

### DETAILED DESCRIPTION

By capturing and analyzing relevant information surrounding and relating to the occurrence of adverse events on a real-time basis, policies and procedures may be implemented to improve patient care and may result in significantly better outcomes.

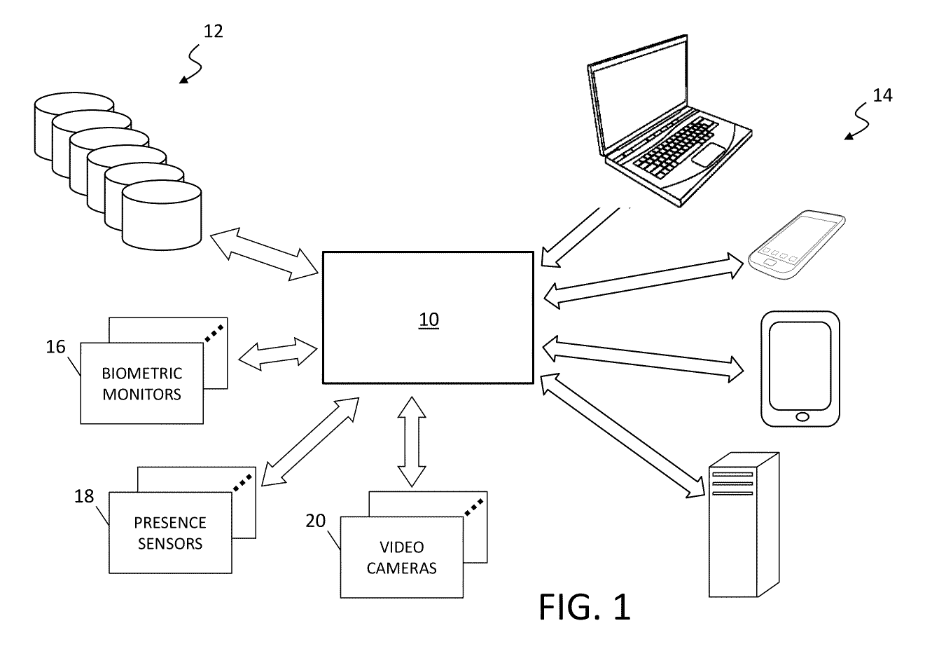

FIG. 1 is a simplified block diagram of an exemplary embodiment of a patient care surveillance system and method 10 according to the present disclosure. The system 10 includes a specially-programmed computer system adapted to receive a variety of clinical and non-clinical data 12 relating to patients or individuals requiring care. The patient data 12 include real-time and near real-time data streams from a variety of data sources including historical or stored data from one or more hospital and healthcare entity databases. Patient data may include patient electronic medical records (EMR), real-time patient event reporting data (e.g., University Health System Consortium PATIENT SAFETY NET), healthcare staff management software data (e.g., McKesson ANSOS), clinical alert, notification, communication, and scheduling system data (e.g., AMCOM software), human capital management software data (e.g., PeopleSoft HR), pharmacy department adverse drug reaction reporting data, etc.

The EMR clinical data may be received from entities such as hospitals, clinics, pharmacies, laboratories, and health information exchanges. This data include but are not limited to vital signs and other physiological data, data associated with comprehensive or focused history and physical exams by a physician, nurse, or allied health professional, medical history, prior allergy and adverse medical reactions, family medical history, prior surgical history, emergency room records, medication administration records, culture results, dictated clinical notes and records, gynecological and obstetric history, mental status examination, vaccination records, radiological imaging exams, invasive visualization procedures, psychiatric treatment history, prior histological specimens, laboratory data, genetic information, physician's notes, networked devices and monitors (such as blood pressure devices and glucose meters), pharmaceutical and supplement intake information, and focused genotype testing.

The patient non-clinical data may include, for example, race, gender, age, social data, behavioral data, lifestyle data, economic data, type and nature of employment, job history, medical insurance information, hospital utilization patterns, exercise information, addictive substance use, occupational chemical exposure, frequency of physician or health system contact, location and frequency of habitation changes, travel history, predictive screening health questionnaires such as the patient health questionnaire (PHQ), personality tests, census and demographic data, neighborhood environments, diet, marital status, education, proximity and number of family or care-giving assistants, address(es), housing status, social media data, and educational level. The non-clinical patient data may further include data entered by patients, such as data entered or uploaded to a social media website.

Additional sources or devices of EMR data may provide, for example, lab results, medication assignments and changes, EKG results, radiology notes, daily weight readings, and daily blood sugar testing results. These data sources may be from different areas of the hospital, clinics, patient care facilities, patient home monitoring devices, and other available clinical or healthcare sources.

Real-time patient data further include data received from patient monitors 16 that are adapted to measure or sense a number of the patient's vital signs and other aspects of physiological functions. These real-time data may include blood pressure, pulse (heart) rate, temperature, oxygenation, and blood glucose level, for example. A plurality of presence sensors 18 are distributed in the facility, such as hospital rooms, emergency department, radiology department, hallways, equipment rooms, supply closets, etc. that are configured to detect the presence of tags or other electronic identifiers so that patient movement and location as well as resource availability and usage can be easily determined and monitored. The presence sensors 18 and tags may be implemented by RFID and/or other suitable technology now known or later developed. Further, a plurality of stationary and mobile video cameras 20 are distributed at various locations in the hospital to enable patient monitoring and identify biological changes in the patient.

The patient care surveillance system 10 receives these patient data, performs analysis, and provides reports and other forms of output data for use by a number of staff, such as physicians, nurses, department chiefs, performance improvement personnel, and hospital administrators. The system 10 may be accessible from a variety of computing devices 14 (mobile devices, tablet computers, laptop computers, desktop computers, servers, etc.) coupled to the system 10 in a wired or wireless manner. These computing devices 14 are equipped to display and present data using easy-to-use graphical user interfaces and customizable reports. The data may be transmitted, presented, and displayed to the clinician/user in the form of web pages, web-based messages, text files, video messages, multimedia messages, text messages, e-mail messages, video messages, audio messages, and in a variety of suitable ways and formats. The clinicians and other personnel may also enter data via the computing devices 14, such as symptoms present at the time of patient in-take, and physician's notes.

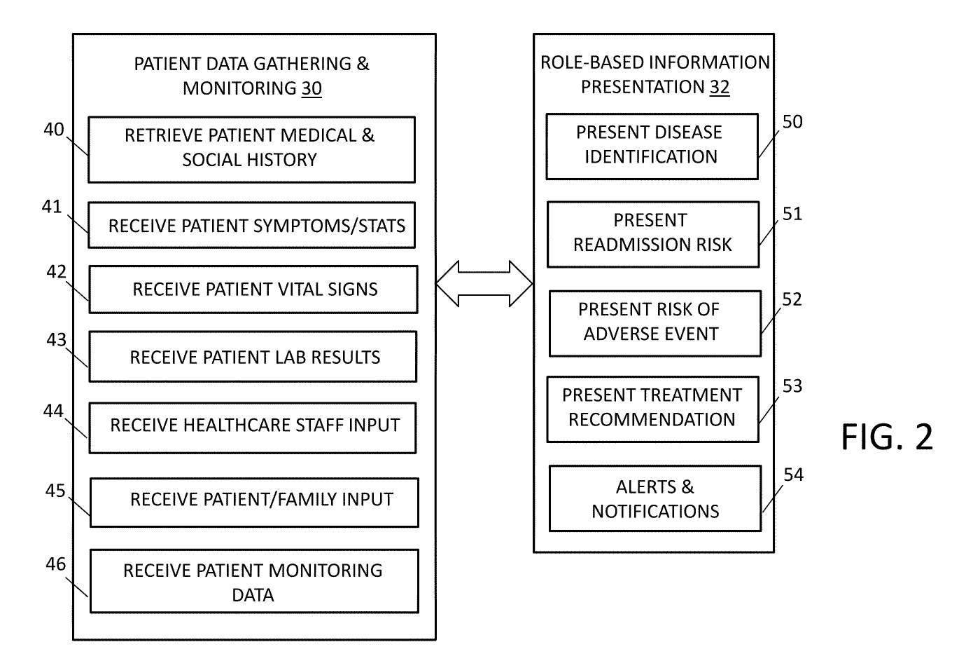

FIG. 2 is a simplified logical block diagram further illustrating the information input 30 and output 32 from the patient care surveillance system and method 10. As noted above, the system 10 retrieves and uses patient data that include real-time and historical pre-existing clinical and non-clinical data 40. When a patient first presents at a medical facility, such as an emergency department of a hospital, his or her symptoms and information 41 such as height, weight, habits (e.g., smoking/non-smoking), current medications, etc. are noted and entered by the medical staff into the system 10. Additionally, the system 10 receives the patient's vital signs 42, such as blood pressure, pulse rate, and body temperature. The healthcare staff may order lab tests and these results 43 are also transmitted or entered into the system 10. The healthcare staff's input 44, including notes, diagnosis, and prescribed treatment are entered into the system 10 as well. Further, the patient and/or family member may be given a tablet computer to enable them to provide input 45 such as comments, feedback, and current status during the patient's entire stay at the hospital. Additionally, the hospital is equipped with a variety of tools, equipment and technology that are configured to monitor the patient's vital signs, wellbeing, presence, location, and other parameters. These may include RFID tags and sensors, for example. The patient monitoring data 46 from these devices are also provided as input to the patient care surveillance system 10.

These patient data are continually received, collected, and polled by the system 10 whenever they become available and are used in analysis to provide disease identification, risk identification, adverse event identification, and patient care surveillance on a real-time or near real-time basis. Disease identification, risk identification, adverse event identification, and patient care surveillance information are displayed, reported, transmitted, or otherwise presented to healthcare personnel based on the user's identity or in a role-based manner. In other words, a patient's data and analysis is available to a particular user if that user's identity and/or role is relevant to the patient's care and treatment. For example, the attending physician and the nursing staff may access the patient data as well as receive automatically-generated alerts regarding the patient's status, and missed or delayed treatment. An attending physician may only have access to information for patients under his/her care, but an oncology department head may have access to data related to all of the cancer patients admitted at the facility, for example. As another example, the hospital facility's chief medical officer and chief nursing officer may have access to all of the data about all of the patients treated at the facility so that innovative procedures or policies may be implemented to prevent or minimize adverse events.

The information presented by patient care surveillance system 10 preferably includes an identification of one or more diseases 50 that the patient has, whether the patient is at risk for readmission due to a particular condition 51, and whether there is a risk of the occurrence of one or more adverse events 52. The system 10 includes a predictive model that provides treatment or therapy recommendations 53 based on the patient's data (e.g., medical history, symptoms, current vital signs, lab results, and the clinician's notes, comments, and diagnosis), and form the fundamental technology for identification of diseases, readmission risk, and adverse events. The system 10 also outputs various notifications and alerts 54 to the appropriate personnel so that proper or corrective action can be taken regarding the patient's treatment and care.

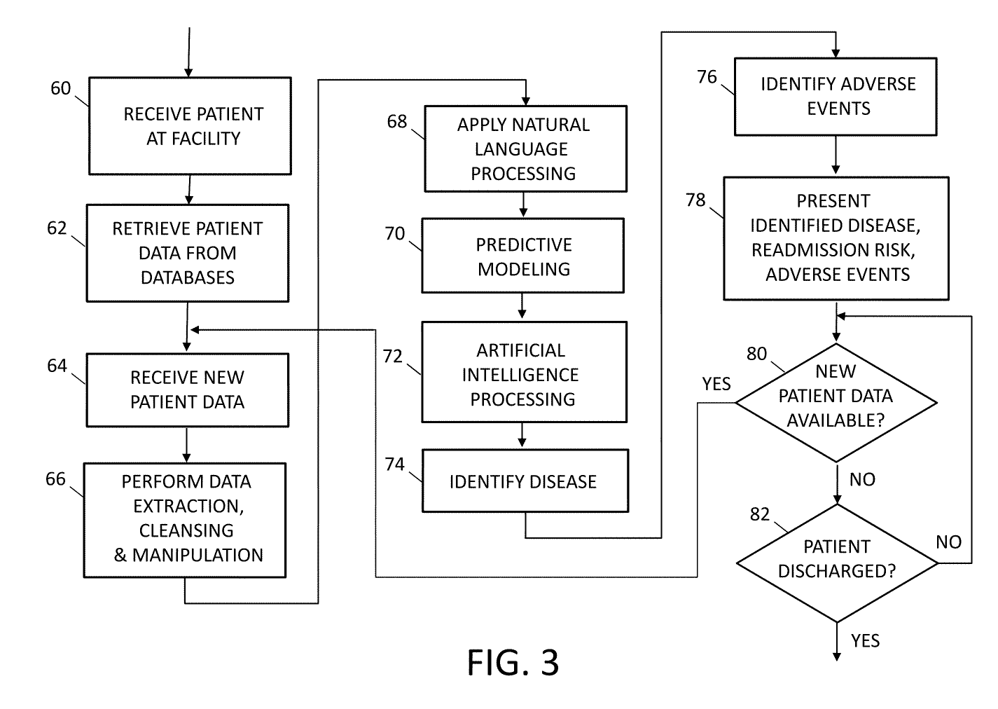

FIG. 3 is a simplified flowchart of an exemplary embodiment of a patient care surveillance system and method 10 according to the present disclosure. FIG. 3 provides an exemplary process in which patient care surveillance is carried out. A patient arrives at a healthcare facility, as shown in block 60. The patient may be brought into an emergency department of a hospital, for example. Upon receiving the patient's identity, the system 10 may immediately retrieve historical data stored in one or more databases related to the patient's medical history, socioeconomic condition, and other information, as shown in block 62. The databases may be on-site at the healthcare facility, or stored elsewhere. The system 10 also begins to receive newly-entered or newly-generated data about the patient, as shown in block 64. The new patient data may include the patient's current symptoms, vital signs, lab results, physician's note and diagnosis, and other data. The system 10 then manipulates or processes the patient data so that they can be usable, as shown in block 66. For example, a data extraction process extracts clinical and non-clinical data from data sources using various technologies and protocols. A data cleansing process “cleans” or pre-processes the data, putting structured data in a standardized format and preparing unstructured text for natural language processing (NLP). The system may also “clean” data and convert them into desired formats (e.g., text date field converted to numerals for calculation purposes).

The patient care surveillance system 10 further performs data integration that employs natural language processing, as shown in block 68. A hybrid model of natural language processing, which combines a rule-based model and a statistically-based learning model may be used. During natural language processing, raw unstructured data such as physicians' notes and reports, may first go through a process called tokenization. The tokenization process divides the text into basic units of information in the form of single words or short phrases by using defined separators such as punctuation marks, spaces, or capitalization. Using the rule-based model, these basic units of information are identified in a meta-data dictionary and assessed according to predefined rules that determine meaning Using the statistical-based learning model, the system 10 quantifies the relationship and frequency of word and phrase patterns and then processes them using statistical algorithms. Using machine learning, the statistical-based learning model develops inferences based on repeated patterns and relationships. The system 10 performs a number of complex natural language processing functions including text pre-processing, lexical analysis, syntactic parsing, semantic analysis, handling multi-word expression, word sense disambiguation, and other functions.

For example, if a physician's notes include the following: “55 yo m c h/o dm, cri. now with adib rvr, chfexac, and rle cellulitis going to 10 W, tele.” The data integration logic (data extraction, cleansing, and manipulation) is operable to translate these notes as follows: “Fifty-five-year-old male with a history of diabetes mellitus, chronic renal insufficiency now with atrial fibrillation with rapid ventricular response, congestive heart failure exacerbation and right lower extremity cellulitis going to 10 West on continuous cardiac monitoring.”

The patient care surveillance system 10 employs a predictive modeling process that calculates a risk score for the patient, as shown in block 70. The predictive model process is capable of predicting the risk of a particular disease or condition of interest for the patient. The predictive model processing for a condition such as congestive heart failure, for example, may take into account a set of risk factors or variables, including the worst values for vital signs (temperature, pulse, diastolic blood pressure, and systolic blood pressure) and laboratory and variables such as albumin, total bilirubin, creatine kinase, creatinine, sodium, blood urea nitrogen, partial pressure of carbon dioxide, white blood cell count, troponin-I, glucose, international normalized ratio, brain natriuretic peptide, and pH. Further, non-clinical factors are also considered such as the number of home address changes in the prior year (which may serve as a proxy for social instability), risky health behaviors (e.g., use of illicit drugs or substance), number of emergency room visits in the prior year, history of depression or anxiety, and other factors. The predictive model specifies how to categorize and weigh each variable or risk factor in order to calculate the predicted probability of readmission or risk score. In this manner, the patient care surveillance system and method 10 are able to stratify, in real-time, the risk of each patient that arrives at a hospital or healthcare facility. Those patients at the highest risk (with the highest scores) are automatically identified so that targeted intervention and care may be instituted.

The patient care surveillance system 10 may further employ artificial intelligence technology in processing and analyzing the patient data, as shown in block 72. An artificial intelligence model tuning process utilizes adaptive self-learning capabilities with machine learning technologies. The capacity for self-reconfiguration enables the system and method 10 to be sufficiently flexible and adaptable to detect and incorporate trends or differences in the underlying patient data or population that may affect the predictive accuracy of a given algorithm. The artificial intelligence model tuning process may periodically retrain a selected predictive model for a given health system or clinic to allow for the selection of a more accurate statistical methodology, variable count, variable selection, interaction terms, weights, and intercept. The artificial intelligence model tuning process may automatically (i.e., without human supervision) modify or improve a predictive model in three exemplary ways. First, it may adjust the predictive weights of clinical and non-clinical variables. Second, it may adjust the threshold values of specific variables. Third, the artificial intelligence model tuning process may evaluate new variables present in the data feed but not used in the predictive model, which may result in improved accuracy. The artificial intelligence model tuning process may compare the observed outcome to the predicted outcome and then analyze the variables within the model that contributed to the incorrect outcome. It may then re-weigh the variables that contributed to this incorrect outcome, so that in the next iteration those variables are less likely to contribute to a false prediction. In this manner, the artificial intelligence model tuning process is adapted to reconfigure or adjust the predictive model based on the specific clinical setting or population in which it is applied. Further, no manual reconfiguration or modification of the predictive model is necessary. The artificial intelligence model tuning process may also be useful to scale the predictive model to different health systems, populations, and geographical areas in a rapid timeframe.

After the data has been processed and analyzed by the foregoing methods, the system and method 10 identifies one or more diseases or conditions of interest for the patient, as shown in block 74. The disease identification process may be performed iteratively over the course of many days to establish a higher confidence in the disease identification as the physician becomes more confident in the diagnosis. New or updated patient data may not support a previously identified disease, and the system would automatically remove the patient from that disease list.

In block 76, the patient care surveillance system and method 10 also identifies one or more adverse events that may become associated with the patient. Adverse events that are at the risk of occurring may be determined by identifying the existence of certain predetermined key criteria. These key criteria, represented by key words, conditions, or procedures in the collection of patient data are triggers that can be indicative of an adverse event. The following are exemplary key words, conditions, or procedures that may be screened and detected for adverse event analysis and determination:

Transfusion of blood products—may be indicative of excessive bleeding, unintentional trauma of a blood vessel.

Cardiac or pulmonary arrest intra- or post-operatively.

Need for acute dialysis—may be indicative of drug-induced renal failure or a side effect to a contrast dye for radiological procedure.

Positive blood culture—may be indicative of a hospital-associated infection.

CT scan of the chest or Doppler studies of the extremities—may be indicative of deep vein thrombosis or pulmonary embolism post-operatively.

Decrease in hemoglobin or hematocrit may be indicative of use of blood-thinning medications or a surgical misadventure.

A fall—may be indicative of a medication adverse effect, equipment failure, or inadequate staffing.

Pressure ulcers.

Readmission within 30 days of discharge following surgery—may be indicative of a surgical site infection or venous thromboembolism.

Restraint use—may be indicative of confusion from medication.

Hospital acquired infections—may be indicative of infections associated with procedures or devices.

In-hospital stroke—may be indicative of a condition associated with a surgical procedure or administration of an anticoagulation.

Transfer to a higher level of care—may be indicative of deteriorating conditions attributed to an adverse event.

Any complication from a procedure.

Some adverse events are related to administration of medications. Therefore, the system 10 may screen the following conditions for further analysis:

Clostridium difficile positive stool—may be indicative of intestinal disease in response to antibiotic use.

Elevated Partial Thromboplastin Time (PTT)—may be indicative of an increased risk of bleeding or bruising.

Elevated International Normalized Ratio (INR)—may be indicative of an increased risk of bleeding.

Glucose less than 50 mg/dl—may be indicative of incorrect dosing of insulin or oral hypoglycemic medication

Rising blood urea nitrogen (BUN) or serum creatinine over baseline—may be indicative of drug-induced renal failure.

Vitamin K administration—may be indicative of bleeding, bruising, or need for urgent surgical intervention

Diphenhydramine (Benadryl) administration—may be indicative of allergic reactions to drugs or blood transfusion.

Romazicon (Flumazenil) administration—may be indicative of benzodiazapene overdoes.

Naloxone (Narcan) administration—may be indicative of narcotic overdose.

Anti-emetic administration—may be indicative of nausea and vomiting that may interfere with feeding, require dosing adjustments with certain medications such as insulin, or delay recovery and/or discharge.

Hypotension or lethargy—may be indicative of over-sedation (sedative, analgesic, or muscle relaxant).

Abrupt medication stop or change—may be indicative of adverse drug reaction or change in clinical condition.

Some adverse events are related to surgical procedures. Therefore, the system 10 may screen the following conditions for further analysis:

Return to surgery—may be indicative of infection or internal bleeding following a first surgery.

Change in procedure—post-operative notes show a different procedure from pre-operative notes which may be indicative of complications or device failure during surgery.

Admission to intensive care post-operatively—may be indicative of an intra-operative or post-operative complication.

Continued intubation, reintubation or use of non-invasive positive pressure ventilation in the post anesthesia care unit (PACU)—may be indicative of respiratory depression as a result of anesthesia, sedatives, or pain medication.

X-ray intra-operatively or in post anesthesia care unit—may be indicative of retained items or devices.

Intra- or post-operative death.

Mechanical ventilation greater than 24 hours post-operatively.

Intra-operative administration of epinephrine, norepinephrine, naloxone, or romazicon—may be indicative of clinical deterioration or over-sedation.

Post-operative increase in troponin levels—may be indicative of a post-operative myocardial infarction.

Injury, repair, or removal of organ during operative procedure—may be indicative of accidental injury if not planned procedure.

Occurrence of any operative complication—e.g., pulmonary embolism (PE), deep vein thrombosis (DVT), decubiti, myocardial infraction (MI), renal failure.

Some adverse events are related to the Intensive Care Unit (ICU). Therefore, the system 10 may screen the following conditions for further analysis:

Hospital-acquired or ventilator associated pneumonia.

Readmission to ICU.

In-ICU procedure.

Intubation or reintubation in ICU.

Some adverse events are associated with perinatal cases. Therefore, the system 10 may screen the following conditions for further analysis:

Parenteral terbutaline use—may be indicative of preterm labor.

3rd or 4th degree laceration.

Platelet count less than 50,000—may be indicative of increased risk of bleeding or bruising requiring blood transfusion.

Estimated blood loss greater than 500 ml for vaginal delivery, or greater than 1,000 ml for caesarean delivery—may be indicative of complications during delivery.

Specialty consult—may be indicative of injury or other harm to a specific organ or body system.

Administration of oxytocic agents post-partum—may be indicative of post-partum hemorrhage or failure of a pregnancy to progress.

Instrumented delivery—may increase the risk of potential injury to mother and baby.

Administration of general anesthesia—may be indicative of rapid clinical deterioration.

Some adverse events are associated with care provided in the emergency department. Therefore, the system 10 may screen the following conditions for further analysis:

Readmission to the emergency department within 48 hours—may be indicative of drug reaction, infection, disease progression, etc.

Time in emergency department greater than 6 hours—may be indicative of excess capacity or lack of inpatient beds, resource or personnel misallocation, or other department failures (e.g., radiology or laboratory system not working)

The patient care surveillance system and method 10 comprise a model that is adapted to predict the risk of particular adverse events, such as sepsis, which is a “toxic response to infection” that has a nearly 40% mortality rate in severe cases. For example, the predictive model for sepsis may take into account a set of risk factors or variables that indicate a probability of occurrence associated with a patient. Further, the analysis may consider non-clinical factors, such as the level of nurse staffing in a unit. In this manner, the system 10 is able to stratify, in near real-time, the risk of patients experiencing an adverse event before it occurs so that proactive preventative measures may be taken.

Referring to block 78 in FIG. 3, the disease identification, risk for readmission, and adverse events are accessible by or presented to healthcare personnel. The presentation of the data may be in the form of periodic reports (hourly, daily, weekly, biweekly, monthly, etc.), alerts and notifications, or graphical user interface display screens, and the data may be accessible or available via a number of electronic computing devices. Many healthcare staff, such as physicians, nurses, department chiefs, performance improvement personnel, and hospital administrators have secured access to reporting and notification provided by the patient care surveillance system 10. The type of data accessible to each user may be tailored to the role or position each user holds in the healthcare facility. For example, a nurse may have access to fewer types of reports than is available to a department chief or hospital administrator, for example.

As a first example, the hospital CEO would like access to a report on the number of patients who had unplanned returns to the operating room during a hospital encounter. He/she may log onto a web-based graphical interface of the patient care surveillance system 10. The CEO is greeted with a screen which displays summary data about an up-to-date tally of patient safety events today. The CEO may click a link to the report function, which enables the user to customize the report by selecting the adverse event of interest (e.g., return to operating room, sepsis, deep vein thrombosis, adverse drug event, etc.), time frame (e.g., year to date, calendar year, fiscal year, month), and unit (e.g., hospital wide, floor, unit, service). He/she can drill down into the individual events to find more granular information about the patient and event.

As a second example, the ICU chief wants to know about use of an order set for their patients who have had a post-operative deep vein thrombosis (DVT). He/she may log onto a web-based graphical interface of the patient care surveillance system 10. He/she may select a report link which enables the user to customize the report by selecting the event of interest (e.g., return to operating room, sepsis, deep vein thrombosis, adverse drug event, etc.), time frame (e.g., year to date, calendar year, fiscal year, month), and unit (e.g., hospital wide, floor, unit, service). The ICU chief may select a report card page, which enables the user to select and see the ICU's performance for DVT prophylaxis and order set compliance. He/she can drill down into the individual events to find more granular information about the patient and event.

As a third example, the attending physician wants to know what high risk events that patients under his/her care are at risk for and if all of the appropriate order sets have been used to mitigate that risk. He/she may log onto a web-based graphical user interface of the patient care surveillance system 10. He/she may be greeted with a default view for his/her patient list which shows hospital data for today (e.g., the number of patient safety events, hospital census, etc.). The user may click a link to the report function that enables the user to select the event of interest (e.g., return to operating room, sepsis, deep vein thrombosis, adverse drug event, etc.), time frame (e.g., year to date, calendar year, fiscal year, month), and unit (e.g., hospital wide, floor, unit, service). He/she can drill down into the individual events to find more granular information about the patient and adverse events.

As another example, an attending physician wants to review his/her performance over the past three months. He/she may log onto a web-based graphical user interface of the patient care surveillance system 10. He/she is greeted with a default view for his/her patient list which shows hospital data for today (e.g., the number of patient safety events, hospital census, etc.). He/she may click a link to the “my patients” function, which enables the user to customize the data by selecting the condition of interest (e.g., laparoscopic cholecystectomy, appendectomy, community acquire pneumonia, etc. . . . ) and time frame (e.g., year to date, calendar year, fiscal year, month). The user can then choose measures of interest (e.g., unplanned return to OR rate, respiratory failure rate, etc.). The user is presented data or reports of those patients with the selected condition of interest and the incidences of the measures of interest along with benchmarks for the hospital and nation, if applicable.

The patient care surveillance system 10 is configured to present or display exemplary drill down report data items that include the following:

Drill Down Report Generic Characteristics:
Patient name
Patient Age
Patient Admitting Diagnosis
Patient Comorbidity
Event (Date/Time/Location)
Event Type
Patient Acuity Score
# of high risk medications
# and type of procedures during hospital encounter
# indwelling lines/catheters and # line days
Provider attribution (Attending, Resident, RN, LPN, MA)

Provider Training Level (if applicable)
Nurse Staffing Ratio
Nurse Tasks List/Burden
Patient Census
Admissions (i.e. flow rate)
Specific fields for each metric in the report may include:
For post-operative DVT/PE:
On appropriate DVT prophylaxis (Heparin, Lovenox, SCDs, IVC
Filter)
Order set use
History of DVT (patient)
For post-operative sepsis:
On antibiotics (type, duration)
Blood Cx sent
For post-operative shock:
Site of bleeding?
I/O for last 24 hours by shift
For unplanned return to surgery:
Site of bleeding
I/O for last 24 hours by shift
For respiratory failure:
Medications
ABG
For shock:
Site of bleeding?
I/O for last 24 hours by shift
For Sepsis (Not POA):
On antibiotics (type, duration)
Blood Cx sent
For narcan use as a trigger:
Opioid use (type, duration, administration method)
Narcan given in emergency department?
Liver function test (LFTs)
For PTT > 100 as a trigger:
On heparin (administration history)
Baseline PTT
Order set use
LFTs
For INR > 6 as a trigger:
On antibiotics (type, duration)
Anticoagulant use
Hemoglobin
LFTs
For glucose < 50 as a trigger:

On hypoglycemic agent (type, duration)
Signs of systemic infection

Creatinine
Order set use (insulin)

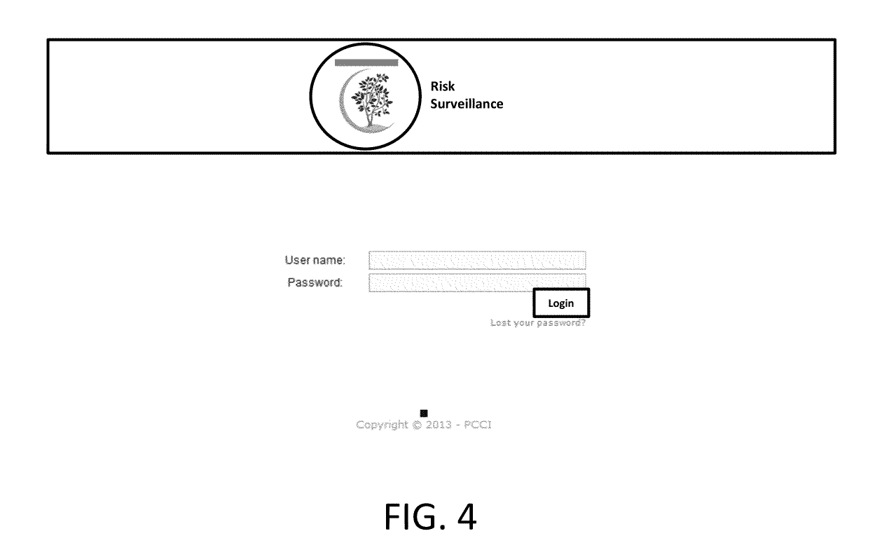

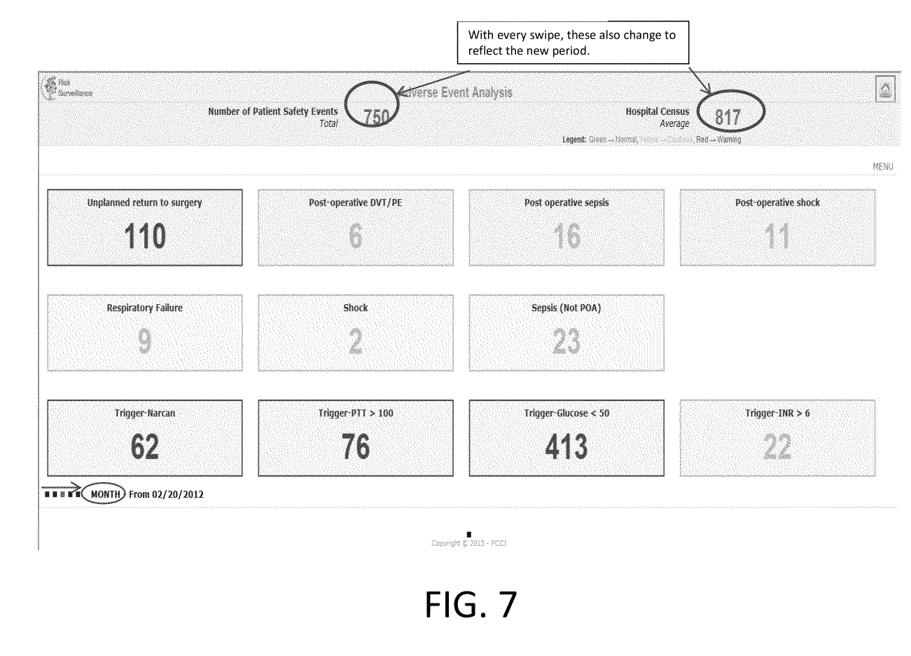

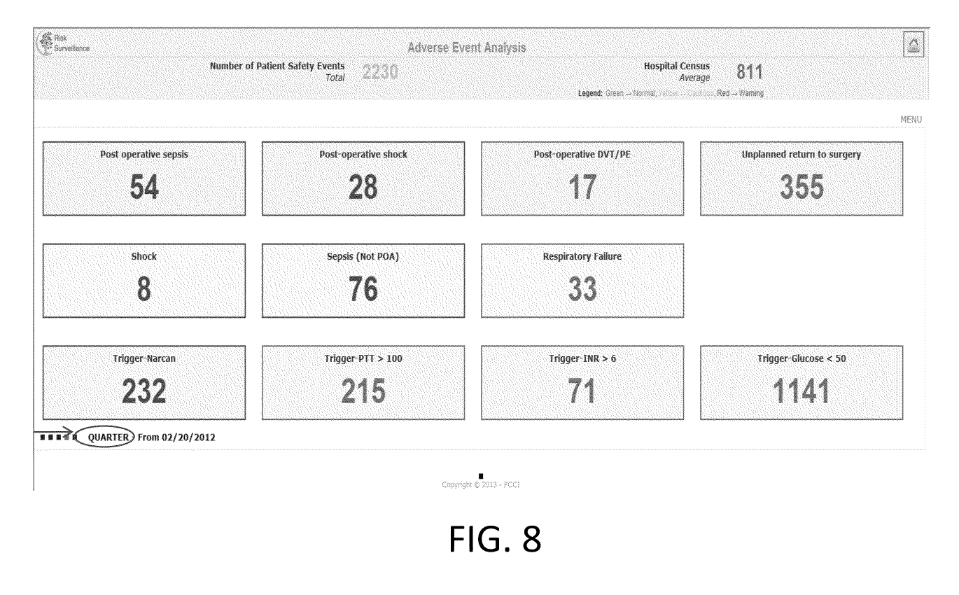

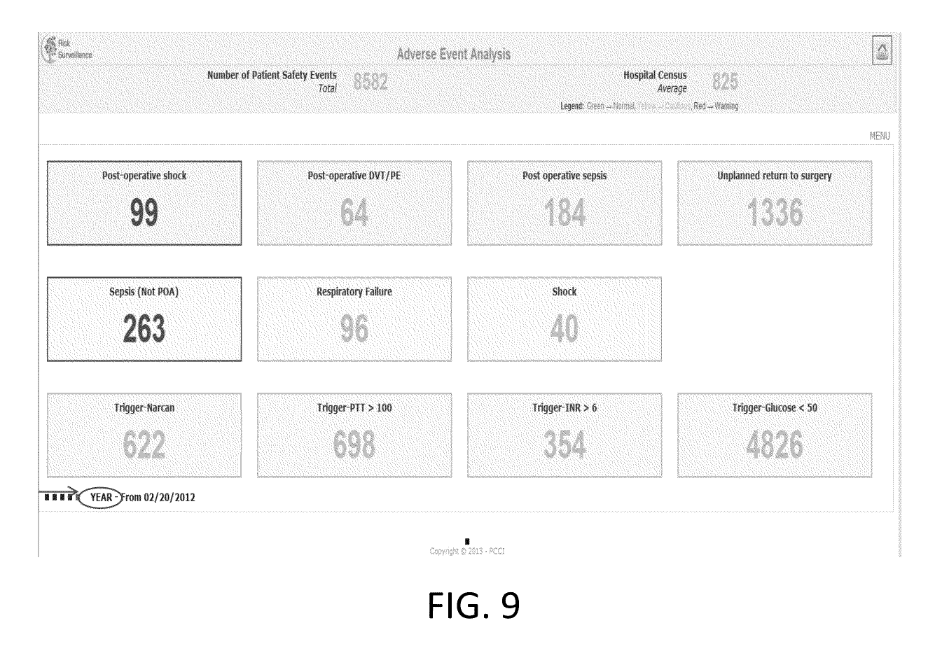

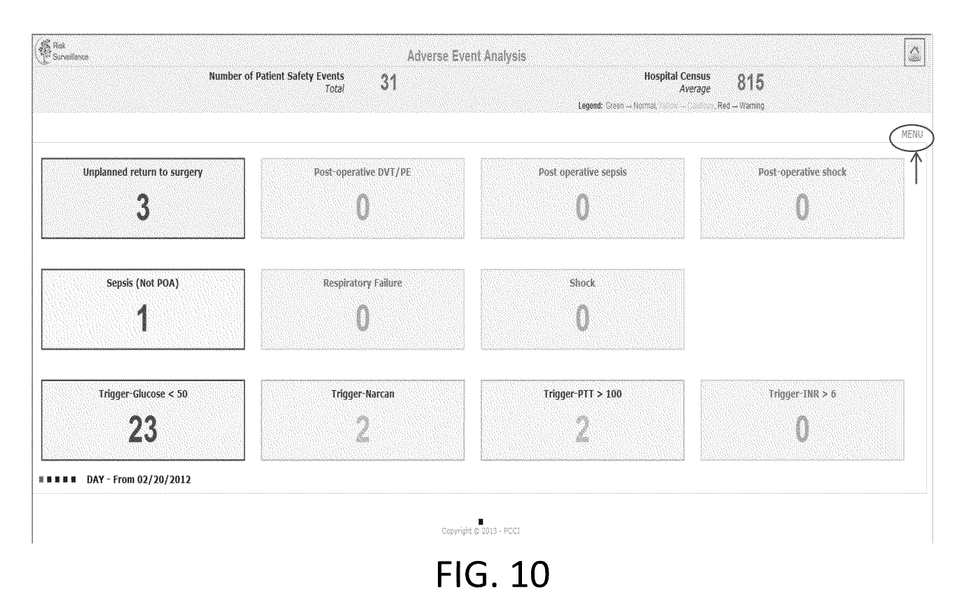

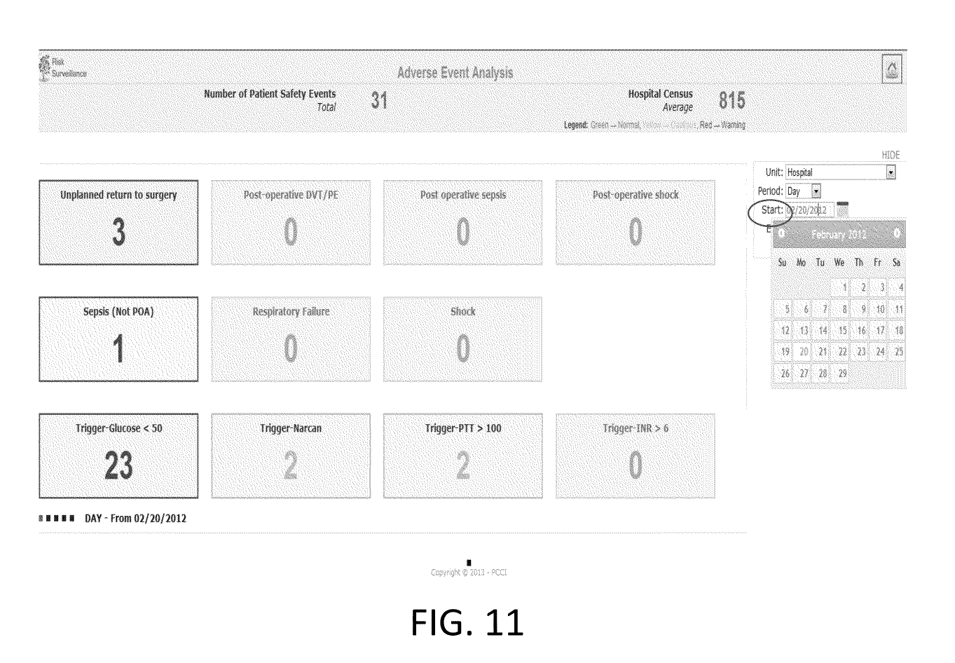

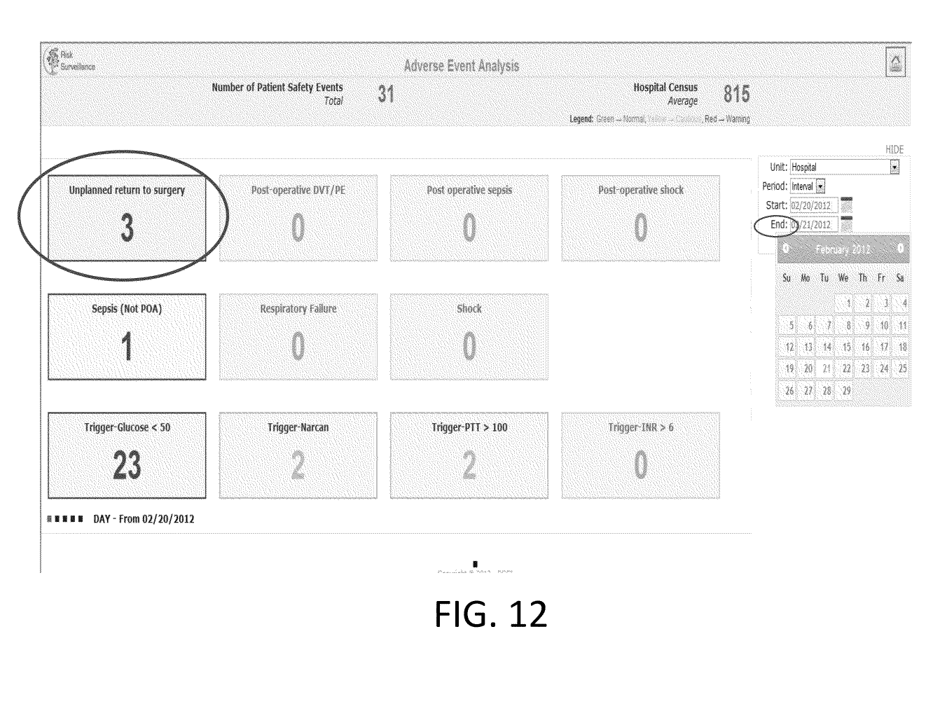

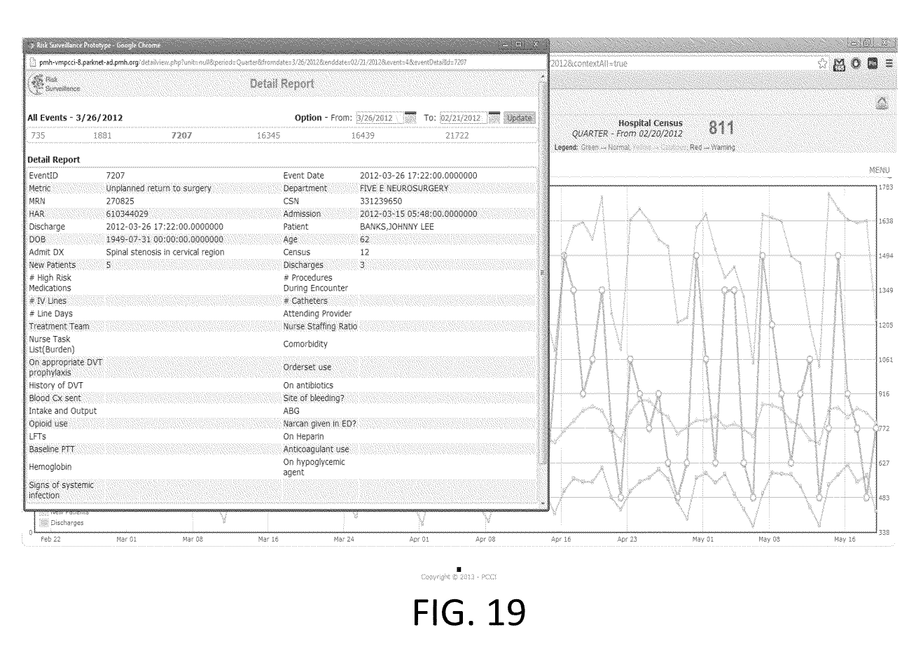

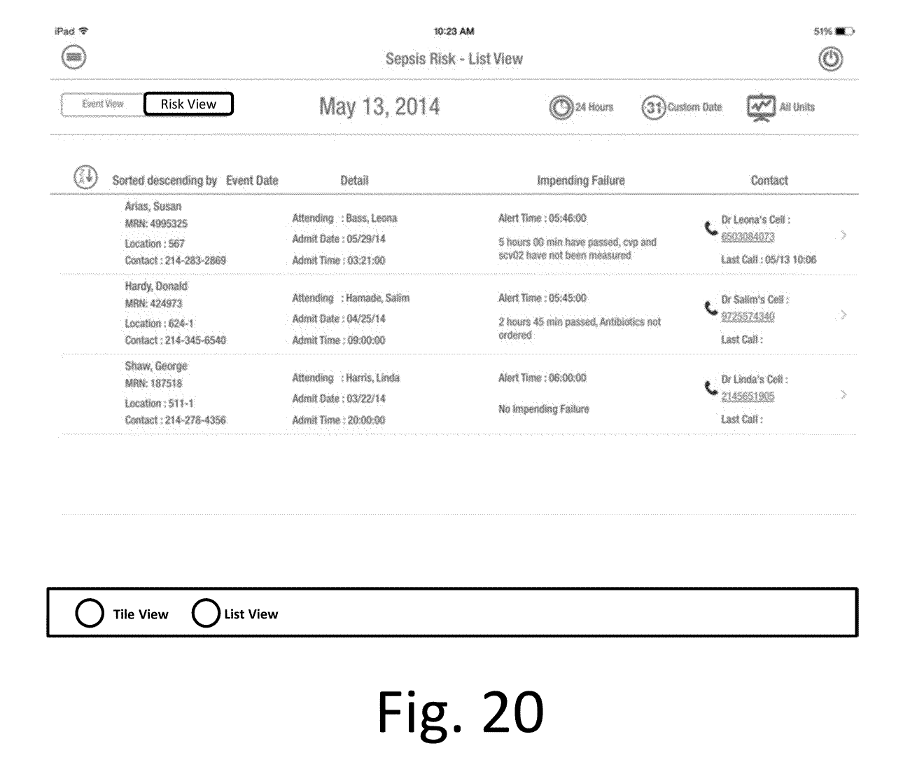

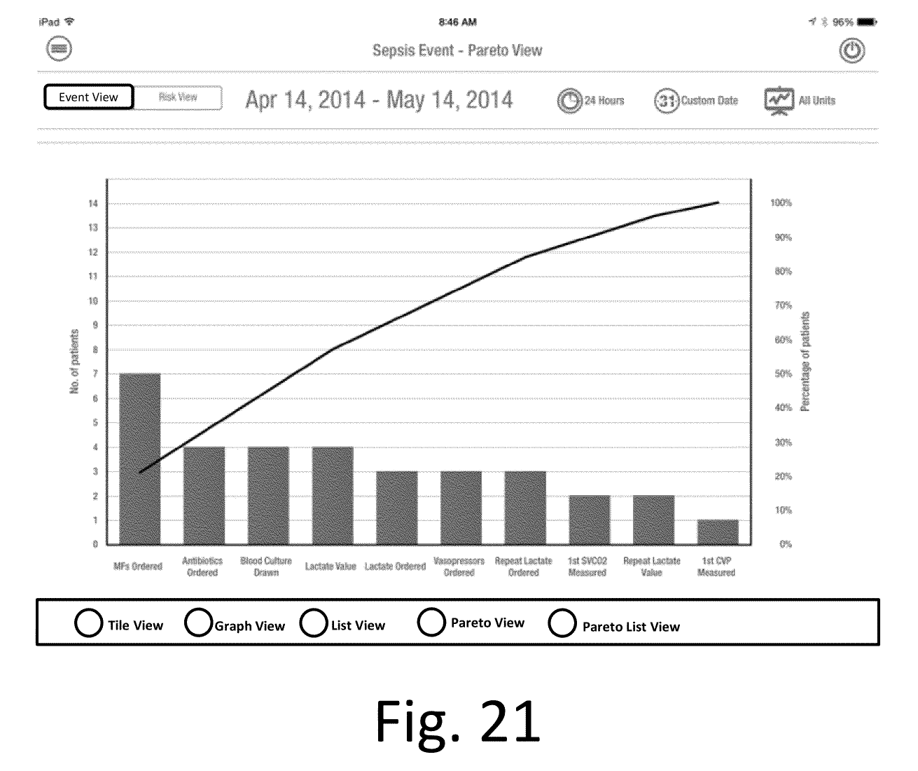

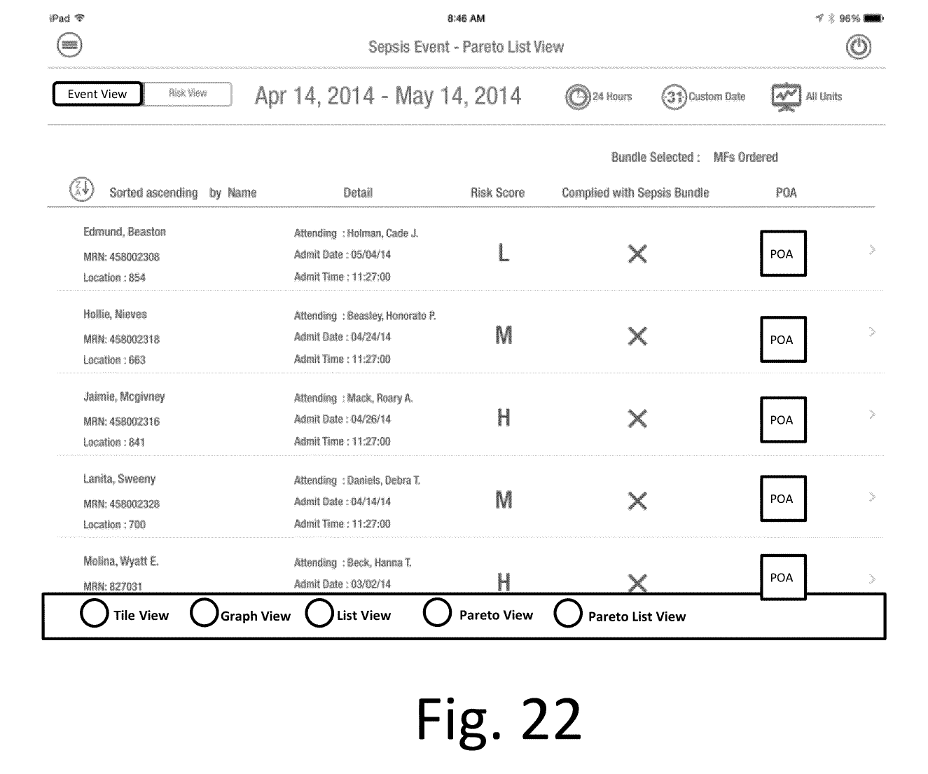

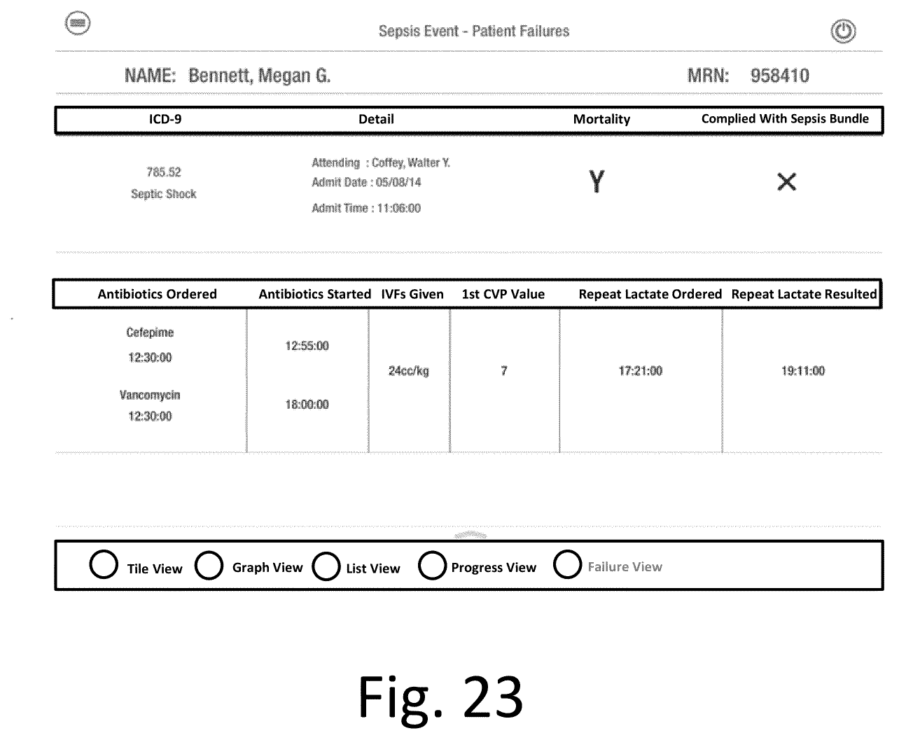

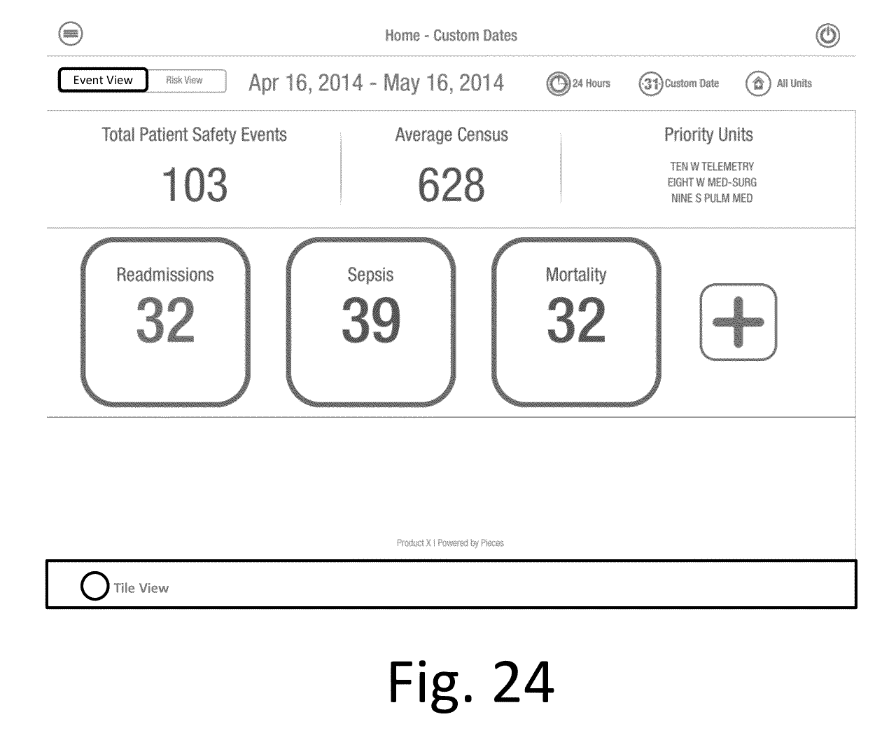

FIGS. 4-25 are exemplary screen displays of a patient care surveillance system and method 10 according to the present disclosure. The system 10 is preferably accessible by a web-based graphical interface or web portal. The figures are shown with annotation that provide explanations of certain display elements.

FIG. 4 is an exemplary secure login page. Upon verifying the user's authorization to access the patient care surveillance system 10, the user is permitted to view and access information related to the user's position or role at the facility. Alternatively, the user is permitted access only to patient data that are relevant to that user, such as an attending physician or nurse having access to those patients under his/her care.

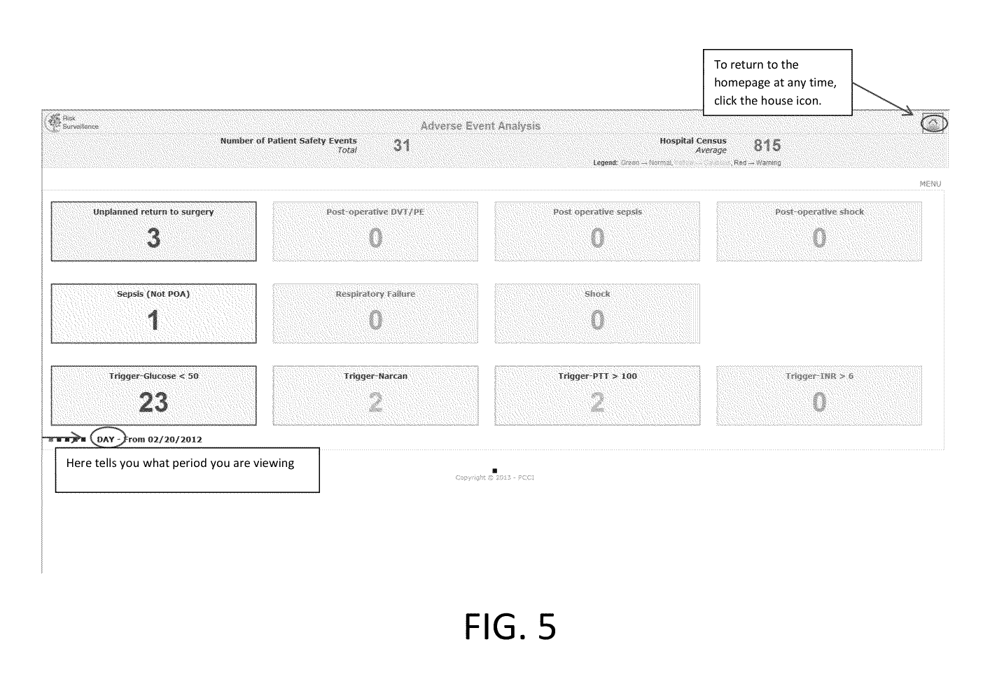

FIGS. 5-25 represent screen shots from the data presentation module of the system. The data presentation module is configured to present a list view, communicating a list of those patients with impending failures on any aspect of the metric under consideration (risk view), or a list of those patients who actually failed on any aspect of the metric under consideration (event view); pareto view, communicating the total number and percentage of actual failures on any aspect of the metric under consideration (event view), or the total number of patients who actually failed on any aspect of the metric under consideration (pareto list view); failure view, communicating only the metric failure(s) encountered by each patient (where applicable); and tile view, communicating the total number of patients with an impending failure for the specific adverse event under consideration (risk view), or the total number of patients who actually failed for each specific adverse event under consideration (event view). For each view, the user can view additional patient information and metric compliance for various time periods.

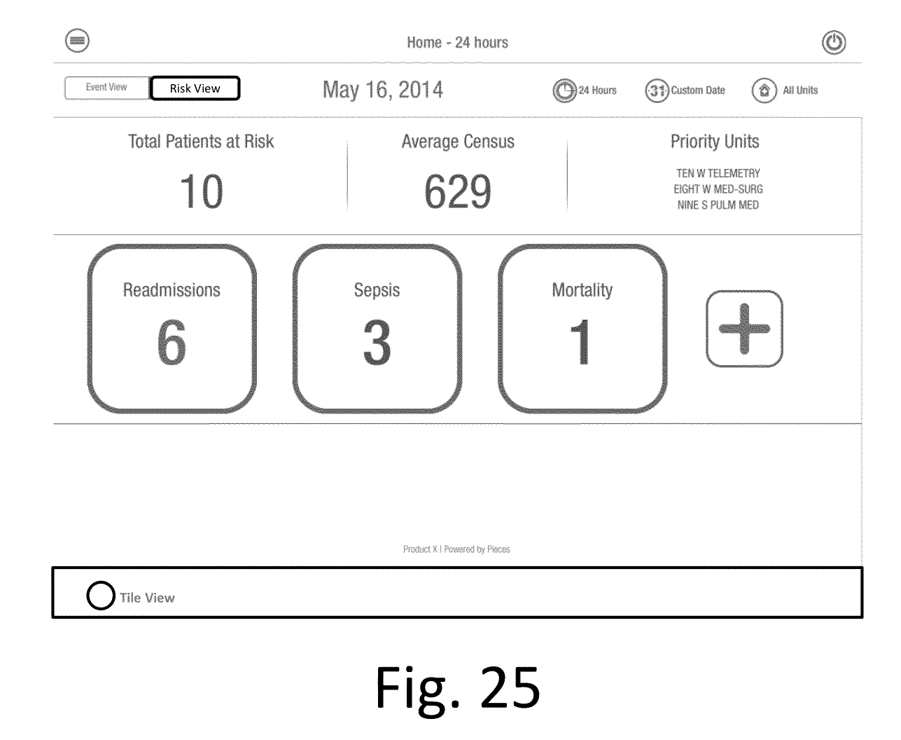

FIGS. 5 and 25 illustrate an exemplary home page or landing page of the patient care surveillance system 10 that gives the user an overview of actual patient safety events over a specified period of time such as 30 days. FIG. 25 illustrates an exemplary home page or landing page of the patient care surveillance system 10 that gives the user an overview of impending patient safety events over a specified period of time such as 24 hours. The exemplary interactive home screen displays the categories for adverse event information relating to a particular type of adverse event, e.g., sepsis that developed within the last 24 hours. A color scheme may be used to highlight certain data. For example, green text may be used to represent normal conditions (i.e., the data are within normal ranges), yellow may be used to represent cautious conditions (i.e., the data are near abnormal ranges and attention is required), and red may be used to represent warning conditions (i.e., the data are within abnormal ranges and immediate action is required).

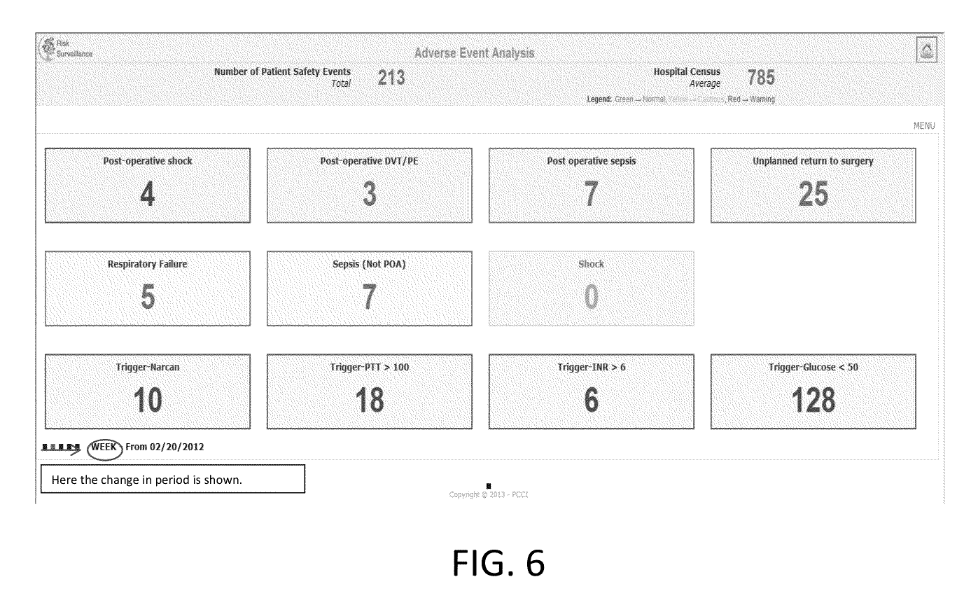

The user may “swipe” to modify the time period to view the number of adverse events that occurred in various time periods (e.g., day, week, month, quarter, year, and specific interval). The user may select an adverse event type (e.g., return to surgery, sepsis, and glucose <50, etc.), the unit (e.g., hospital, floor, unit, emergency department, ICU, etc.), time period (e.g., days, weeks, months, years), context or nurse staffing level, and the report start and end dates. Clicking on any of the adverse events of interest leads to more detailed data in report form or graphical representations. FIGS. 6-12 demonstrate the exemplary screens for various time periods.

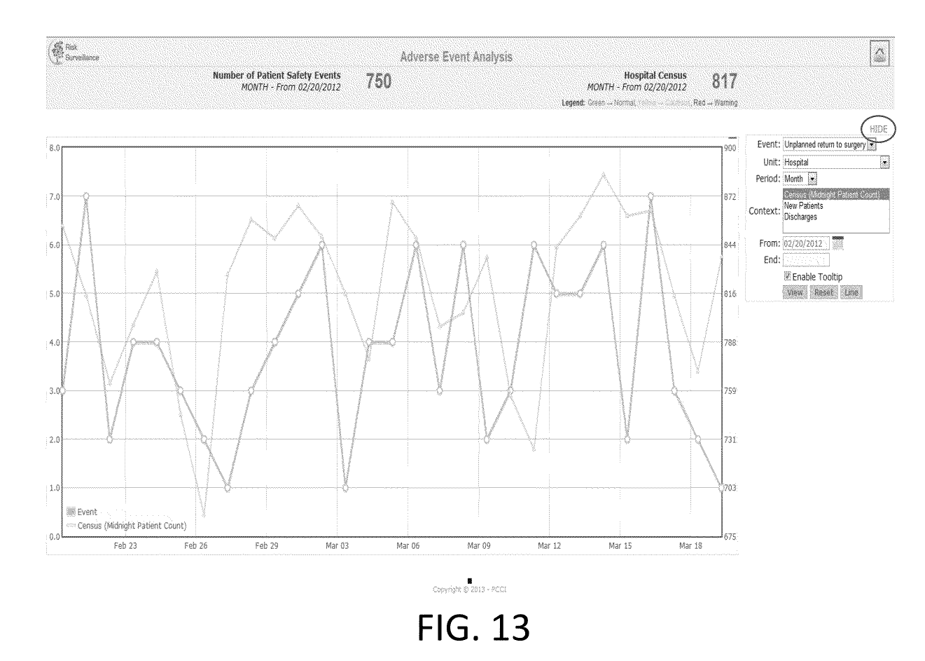

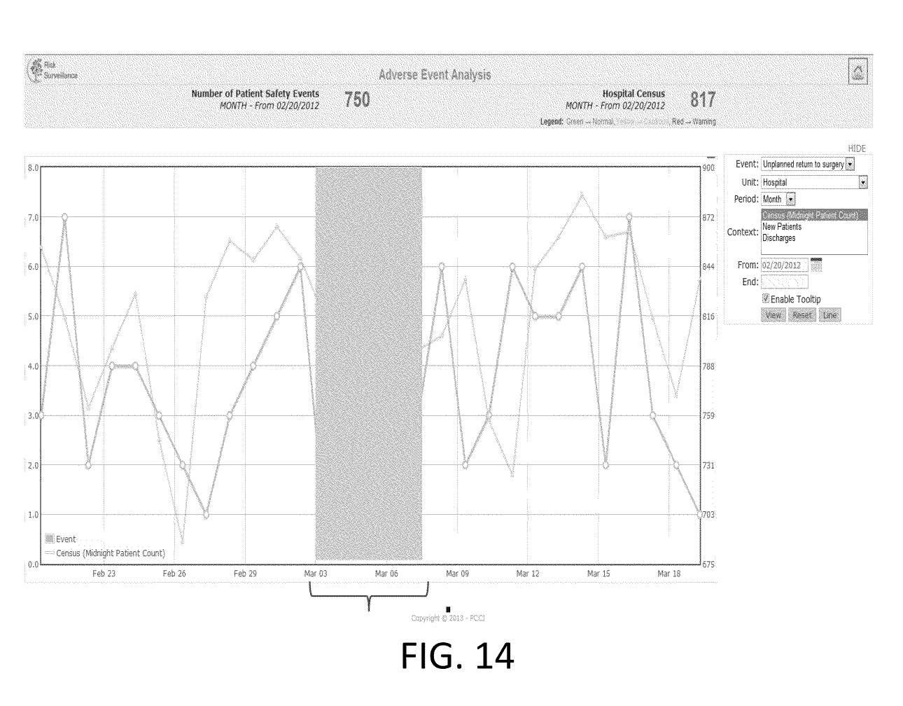

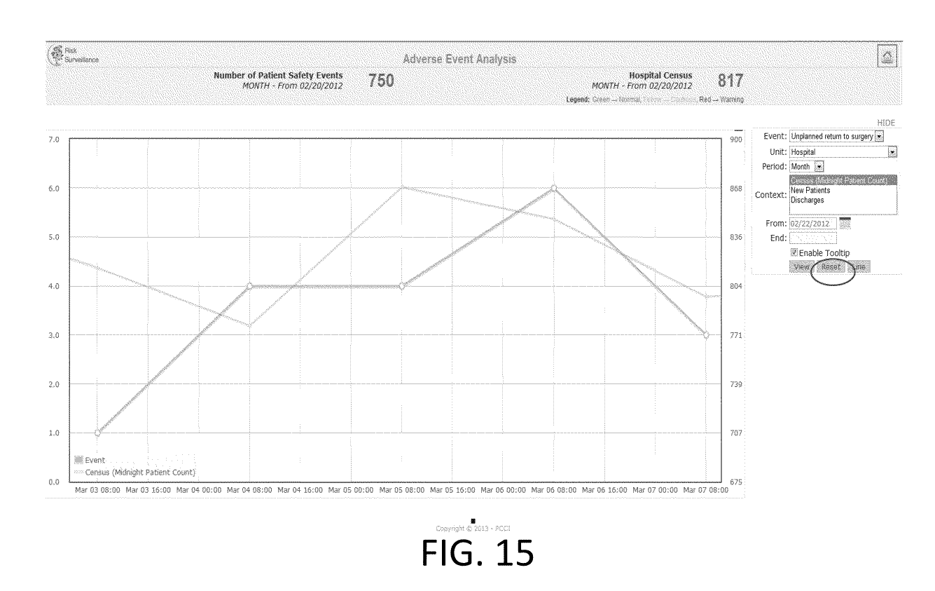

FIGS. 13-19 and 21 are exemplary screens for graphical representations of a particular event in response to the user's selection and input. The exemplary screen may highlight the post-operative DVT/PE, shock, and post-operative shock graphs for ease of viewing. The user may select a more specific timeframe to obtain more detailed information, as shown in FIGS. 14 and 15.

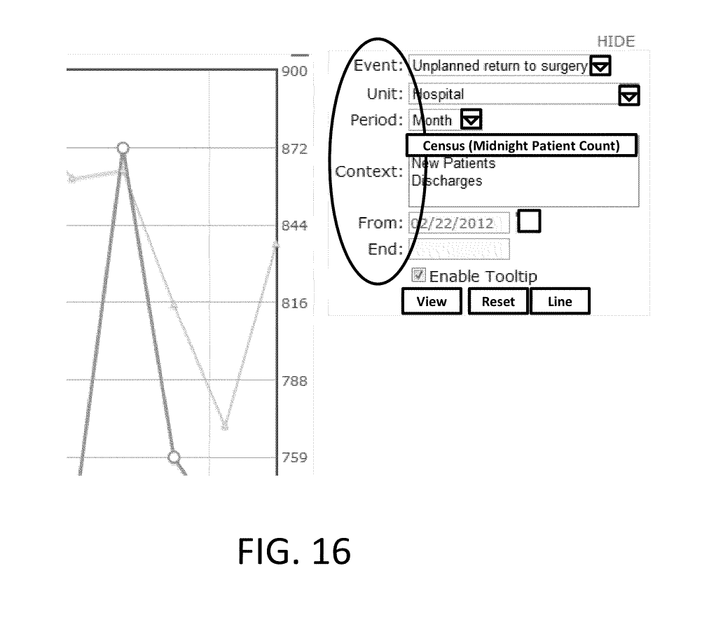

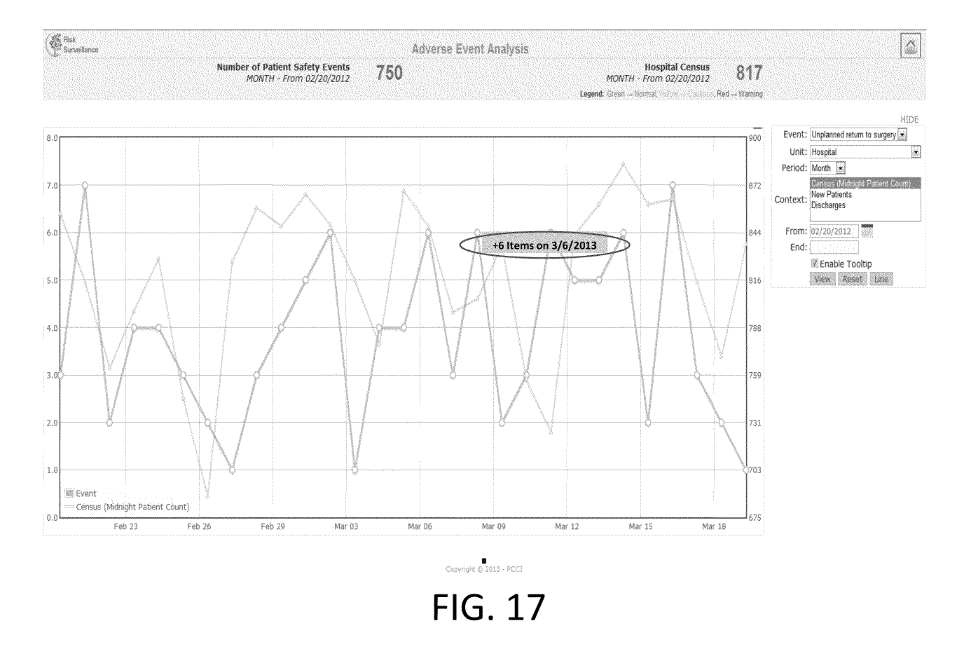

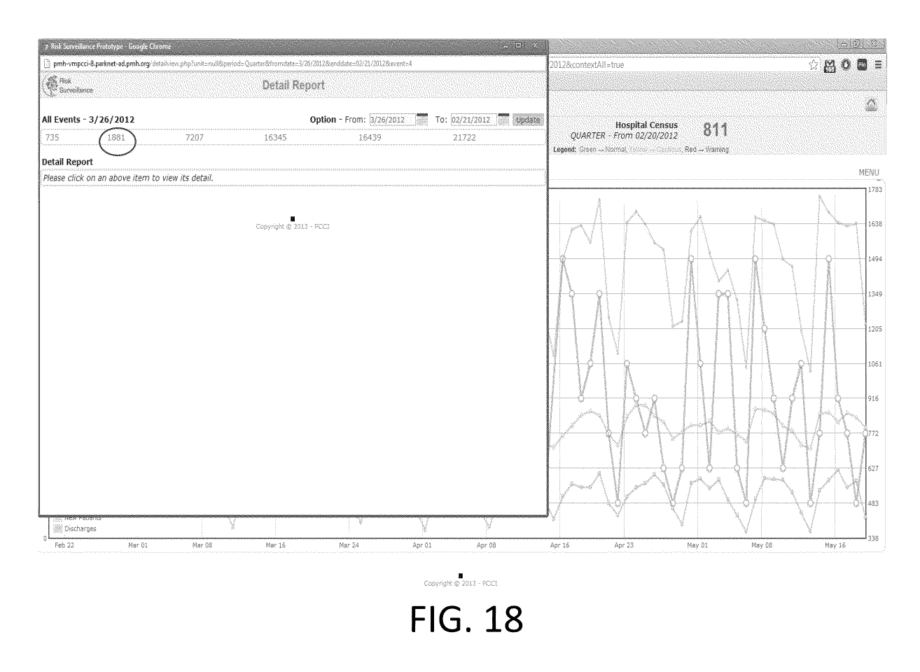

FIG. 16 is a close-up of the exemplary menu pane that may be used to enter or change various parameters or variables to filter the displayed data or graph. For example, the user may specify the event type, unit, context, and time period. On mouse-over, more detailed information about the selected graphical point may be displayed, such as shown in FIG. 17. The user may click on a particular event to drill down for more detailed information of that event. Selected portions of data may be displayed in a more muted fashion to facilitate ease of reading and comprehension. FIGS. 18-20, 22, and 23 demonstrate how a user can drill down to a specific event to obtain a report containing more information about that selected event.

Along with the detection of adverse events or potential adverse events, contextual information associated with the detected event are also collected and analyzed. A contextual variable refers to measures which give insight to surrounding issues or activities that may affect the outcome of interest. For example, the staffing level, hospital census, number of high risk medications, number of new patients, resource availability, location of the patient, and other data may be collected and accessible so that a hospital administrator may be able to determine whether inappropriate nurse staffing levels in a particular unit or floor may be associated with the occurrence of a particular adverse event. The user may select the desired contextual variable(s) to view this information.

The patient care surveillance system and method 10 are further operable to capture, record, track and display whether patients received proper care before and after the occurrence of adverse events, i.e., whether proper steps were taken to avoid an adverse event, and to mitigate injury after an adverse event.

Below are exemplary use cases concerning sepsis, hypoglycemia, and thirty-day mortality adverse events that further highlight and illustrate the operations of the patient care surveillance system and method 10.

Sepsis is a “toxic response to infection” that results in approximately 750,000 cases per year with a nearly 40% mortality rate in severe cases. Due to the rapidly progressive and fatal nature of this condition, early detection and treatment are essential to the patient's survival. The patient care surveillance system and method 10 actively track the clinical status of septic patients in order to provide close monitoring, enhanced clinical decision-making, improved patient health and outcomes, and cost savings.

A first example involves an 80 year-old male with a past medical history of chronic obstructive pulmonary disease (COPD). The patient's medical history indicates that he has been a smoker since the age of 18, and has a weakened immune system due to an autoimmune condition. This patient came to the emergency department complaining of fever (˜103 degrees Fahrenheit when checked by the nurse), with alternating bouts of sweating and shaking chills. He also complained of nausea, severe chest pain and incessant coughing accompanied by bloody and yellow mucus. The patient may enter all of his complaints into a mobile tablet computer that is provided to him by the nurse during triage. The tablet computer provides a graphical user interface displaying an area for the patient to describe all of his complaints, or check off applicable symptoms from a list. Alternatively, the nursing staff may enter the patient's symptoms and complaints into the system along with notes from his/her own observations. The entered data become a part of the patient's electronic medical record (EMR). The attending physician may review all of the available patient data including the past medical history and the patient's symptoms prior to evaluation.

After performing the physical evaluation, the attending physician enters relevant information from his/her own assessment in the EMR, which may be via a graphical user interface on a table computer, a laptop computer, a desktop computer, or another computing device. A predictive model of the patient care surveillance system 10 extracts the available patient data in real-time and immediately performs disease identification. The patient care surveillance system 10 presents or displays to the healthcare staff a disease identification of bacterial pneumonia, and also classifies this patient as high-risk for readmission due to his comorbidities. The attending physician indicates his agreement with the predictive model's disease assessment and enters an order for antibiotics and also requests that a device to monitor the patient's vital signs be placed on his arm. The patient's vital signs are continually measured and transmitted to the patient care surveillance system 10 and recorded as a part of the patient's EMR. The patient is given his medications and is admitted to the intensive care unit (ICU). The patient is also given a device such as a wristband that incorporates an RFID tag that can be detected by sensors located at distributed locations in the hospital, including, for example, the intensive care unit, patient rooms, and hallways.

Six hours following the patient's arrival, the vital sign monitor begins to issue an audible alert, having detected an abnormality. The monitor measures and transmits the patient's current vital signs that indicate the patient's blood pressure is 85/60, pulse is 102, temperature is 35.9 degrees Celsius, and peripheral oxygen saturation (SpO2) is 94% on room air. Based on these vitals measurements, the patient care surveillance system 10 automatically sends an alert in the form of a page, text message, or a voice message, to the charge nurse and the attending physician. The nurse goes to the bedside to evaluate the patient, and the physician orders initial lab tests that may include a complete blood count (CBC), comprehensive metabolic panel (CMP), and lactate levels to confirm his/her initial diagnosis of potential sepsis.

Once the lab results indicating that the patient has findings concerning for sepsis become available and are transmitted or entered into the patient care surveillance system 10, the system 10 automatically issues a sepsis best practice alert (BPA) that is conveyed to the attending physician. As a result, the attending physician places orders from the sepsis order set (3-hour sepsis bundle) for IV fluids (IVFs), blood cultures, and two antibiotics upon receiving the BPA. Thus, the IVFs are started, blood cultures are drawn, and both antibiotics are administered and completed within the first two hours of the BPA. A completion status with a timestamp for each requirement of the 3-hour sepsis bundle protocol is transmitted in real-time to the system 10 and recorded.

In response to the timely treatment, the patient's vitals return to normal, as measured by the vital signs monitor, and the patient's change in clinical status is immediately communicated to the system 10 and recorded. The patient's change in clinical status may trigger or set a flag for evaluation by the medical leadership such as a medical director of the facility. The patient care surveillance system 10 may recommend that the medical director issue an order that the patient be evaluated regularly over the course of the next 24 hours, and that if the patient's vital signs remain normal after the 24-hour evaluation period, the patient is to be transferred from the intensive care unit to a lower level of care to provide room for more critical patients. The medical director accepts the recommendation and enters the order in the system 10.

However, while the patient's vitals remain normal for 24 hours, he remains in the intensive care unit because the order to transfer the patient was inadvertently not carried out. The patient's location is continually monitored and noted by the RFID sensor system and transmitted to the patient care surveillance system 10. The patient's location following the evaluation period is still noted as “ICU” with corresponding timestamps in the system 10. The system 10 may detect and automatically flag this inconsistency between the transfer order and the patient's location for review by the proper personnel. An alert may be issued to notify the appropriate personnel.

The hospital's administrators have access to the patients' data. For example, the hospital administrators may review data associated with patients from the past 30 days that had sepsis non-POA (not present on admission). The hospital administrators may conclude, given the data, that patient transfer orders must be expedited once they ensure that a patient is improving for at least 24 hours. New protocols may be put in place to ensure that the patient transfer from a critical unit is prioritized through improved coordination with physicians, case managers, environmental services, and transfer staff to ensure that sufficient capacity and resources are available for more vulnerable patients. As a result, improvements are made to the hospital's operating efficiency and resource allocations.

In a second example also involving sepsis, the same 80 year-old male with a past medical history of chronic obstructive pulmonary disease (COPD) and identical symptoms as above is taken to the emergency department. The same pneumonia diagnosis is presented by the patient care surveillance system 10 and accepted by the attending physician. Antibiotic treatment is prescribed and administered to the patient accordingly. Six hours after the patient's arrival, a change in the patient's vital signs causes an alert to be sent to the charge nurse and the attending physician. Based on the lab results, sepsis is suspected by the system 10 and the attending physician, and the physician orders the three-hour sepsis bundle for IV fluids, blood cultures, and two antibiotics according to the sepsis best practice alert (BPA). The IVFs are started, blood cultures are drawn, and one of the two antibiotics are administered within the first two hours of the BPA. A status (“completed” or “not complete”) with timestamp for each requirement of the three-hour sepsis bundle protocol is entered into and recorded in the system 10.

In this example, assume that the second antibiotic treatment has not yet been administered, and therefore the status of “not complete” is still associated with the second antibiotic order. When a medical director reviews the patient data in real-time, he/she can easily see that not all of the protocols of the three-hour sepsis bundle have been executed within the required timeframe. He/she can also see that there are 30 minutes remaining before the expiration of the 3-hour time window. The medical director may call, page or send a text message to the patient's physician (for ordering-related issues) or the patient's nurse (for administration-related issues), whose name and contact information are displayed or provided as clickable links in the graphical user interface of the system 10, alerting him/her of the urgency to administer the remaining antibiotic treatment within the next half hour. Alternatively, the system 10 may automatically generate and transmit an alert to healthcare personnel (attending physician and/or nurse) when treatment time windows are near expiration while some of the ordered treatments still have an “incomplete” status. The patient's nurse immediately responds to the message from the medical director and administers the second of two antibiotics prior to the end of the 3-hour time window. The patient's vitals return to normal, as measured by the vitals monitor, and his change in clinical status (i.e., return to normal) is immediately communicated to the system 10 and stored.

In this second sepsis example, real-time information is communicated to the medical director who is capable of alerting members of the treatment team. This is especially relevant for time-sensitive therapies which require a specific time window to avoid additional adverse events. The use of real-time surveillance technology intended for medical leadership facilitates timely adherence to prescribed treatment plans. Improvements in provider care plan compliance may lead to a natural reduction in healthcare costs, as a result of avoiding additional adverse patient outcomes, and a corresponding improvement in population health.

In a third example involving sepsis, a 47-year old man with no known or recorded medical history is taken to the emergency department at 2:26 am complaining of history of “crampy” abdominal pain associated with non-bloody/non-bilious emesis that he has endured for two days. In triage, this patient's vital signs are taken and indicate blood pressure at 92/61, pulse rate at 104, body temperature at 35.9 degrees Celsius, and peripheral oxygen saturation (SpO2) at 94% on room air. The patient's vital signs are entered into the patient care surveillance system 10 along with the symptoms via a graphical user interface. The attending physician orders initial lab tests at 2:40 am that include a complete blood count (CBC), comprehensive metabolic panel (CMP), and peripheral venous blood lactate to confirm his initial diagnosis of potential sepsis. The labs are drawn at 2:47 am, and the results are returned at 3:28 am and entered into the system 10. The lab results indicate that the patient has findings concerning for sepsis, and the sepsis best practice alert (BPA) is issued at 3:29 am by the system 10.

The attending physician accepts the BPA and places orders from the sepsis order set for IV fluids, blood cultures, and antibiotics at 3:30 am. IVFs are started, blood cultures are drawn, and one of the two antibiotics is administered and completed within the first two hours of the patient's hospitalization. The second antibiotic treatment is delayed because the patient was taken to radiology for additional imaging. Therefore, the second antibiotic treatment began at 5:56 am, about 3½ hours after patient's presentation to the emergency department. A status and timestamp for each of the orders in the order set are entered in the system 10 and stored.

An order to take a repeat lactate is also delayed because medical personnel in the ICU are preoccupied with resuscitating another critical patient requiring CPR. The patient care surveillance system 10 issues and automatically transmits a notification of impending failure of the repeat lactate order (as required by the six-hour sepsis bundle metric) to the ICU medical director and/or the attending physician informing them that there is an impending treatment failure for this particular patient. As a result, the attending physician ensures that the repeat lactate is drawn immediately. Subsequently, the vitals monitor automatically measures the patient's vitals, which confirms that the treatment worked and the patient's conditions are reverting back to normal.

As illustrated by this example, patient-related data around adverse events are transmitted in real-time to the patient care surveillance system 10 to communicate patient statistics for adverse events such as sepsis POA (present on admission) across the entire hospital for access by relevant staff. The ready availability of the patient data helps to improve care coordination by giving medical leadership real-time information which can inform institutional policy changes to enhance patient care. Specifically, the retrospective view allows the medical director and chief of infectious diseases, for example, to see that a code blue was a contributing factor associated with not satisfying all of the requirements related to the 6-hour sepsis bundle. The repeat lactate test was delayed. When a medical director or chief of infectious diseases select to view the last 24-hours of patient data provided by the system 10, they may see the number of septic patients with and without fatal outcomes who experienced bundle failures. For example, if the data show that a majority of septic patients experienced some form of failure with the execution of the order set within the required time window, the medical leadership may realize a need to augment the medical staff to ensure that competing priorities do not impact timely administration of treatment orders.

In a fourth example involving sepsis, the same 47-year old man with no known or recorded medical history is at the emergency department at 2:26 am with the same symptoms, vitals, and lab results as described above. The lab results indicate that the patient has findings concerning for sepsis, and the sepsis best practice alert (BPA) is issued at 3:29 am by the system 10. Similar to the above example, the three-hour sepsis order set was prescribed; the second antibiotic was not administered because the patient was taken from the ED to radiology for imaging.

A status and timestamp for each element of the sepsis bundle are available for access by certain healthcare personnel, including hospital administrators. Upon viewing the status of each intervention, a hospital administrator notices that the second antibiotic treatment is still not administered and that the patient's current location shows that he is in the radiology department. The administrator may immediately deploy resources to expedite transfer of the patient back to the emergency department in order to complete the administration of the second antibiotic before the 3-hour window expires.

As a result of real-time notification relaying information regarding a potential delay in antibiotic administration, clinical leadership is able to take the necessary steps to ensure that resources were sufficient and the patient is in a place to receive timely treatment. The system 10 thus facilitated improved patient outcomes and ultimately containing costs associated with additional adverse outcomes.

Hypoglycemia is defined by abnormally low blood glucose levels. Standard “low” threshold is quantified as less than 70 mg/dL. The adverse consequences of hypoglycemia include seizures, permanent brain damage, or loss of consciousness (due to insulin shock). As a result of the potentially fatal adverse outcomes associated with this condition, a tool to monitor patient glucose levels is critical to identify and prioritize individuals who need therapy in an expedited manner. As a further example illustrating the operations of the patient care surveillance system and method 10, a 78-year old Asian female with a history of diabetes comes to the emergency department complaining of dizziness when standing, and has experienced shakiness and headaches on and off for the past three days. This patient is found to have a blood glucose level <50 mg/dL, confirming hypoglycemia. This diagnosis is facilitated by a subcutaneous glucose sensor that measures the patient's blood glucose levels. The glucose monitoring sensor is operable to automatically transmit the measured glucose levels to the patient care surveillance system 10 that stores the data as a part of the patient's electronic medical record (EMR).

Information about the patient is collected by the patient care surveillance system 10 and made available to the chief of endocrinology. When the chief sees the patient's information via the graphical user interface of the system 10, he requests an immediate page to be sent to the attending physician requesting immediate medication therapy for this patient. As a result of the page, the attending physician immediately enters the order in the system, and notes its urgency. When the therapy is ready, it undergoes a verification process requiring two nurses to check the medication before it is administered to the patient to avoid medication error. The hospital's medical leadership instituted the two-check verification policy as a new hospital-wide medication evaluation protocol with the aim of reducing medication errors. The nursing staff who performs the checks must note the checks and their identities in the patient care surveillance system 10. After administering the medication, the patient's blood glucose level returns to normal and her dizziness, shakiness, and headache subside.

The patient's information, when entered into the EMR, is automatically available for viewing immediately via the graphical user interface of the patient care surveillance system 10. The system 10 gives the medical staff and leadership the opportunity to perform real-time patient tracking and monitoring, as well as to identify patients experiencing adverse events in real-time. The availability of real-time adverse event information significantly reduces the likelihood that a patient experiencing an adverse event will be left untreated. Further, if the adverse event progresses without appropriate clinical attention, the system 10 issues automatic alerts or notifications to the appropriate personnel so that corrective action can be taken before an irreversible outcome occurs.

In addition, the availability of patient data gives medical staff and leadership the ability to spot patient care issues that should be addressed. For example, patient data over a 60-day period may reveal that a large percentage of hypoglycemic patients experience some type of medication error, and that a large percentage of those patients suffer fatal outcomes. Due to the significance of the medication error in hypoglycemic patients, a new protocol requiring two medication checks is instituted to reduce the occurrence of these incidents.

Thirty-day mortality is a quality metric which is incorporated in multiple national reporting programs to assess hospital performance. Outcome measures, such as mortality rates, are considered reliable metrics to evaluate hospital performance because they fully capture the end result of healthcare. As such, in order to align institutional priorities with national quality-related priorities, many organizations emphasize the development and implementation of solutions aimed at reducing mortality rates. In this example, a 70-year old obese male is admitted overnight to the hospital with severe chest pain and shortness of breath. The physician decides to keep the patient overnight for monitoring since the patient suffered from a mild heart attack eight months ago. Additionally, the patient has a family history of coronary artery disease and arrhythmias, and the patient has high blood pressure, high cholesterol, and diabetes. The attending physician orders an electrocardiogram (ECG) and cardiac enzyme tests for the patient to assess for heart damage and a possible myocardial infarction. While awaiting completion of these tests, the patient develops shortness of breath and palpitations, and he becomes hypotensive. The rapid assessment team (RAT) who received no prior notification of this patient's status, arrives while the ECG is being performed which confirms the presence of a heart attack. The patient is immediately transported to the cath lab, but intervention is delayed because all members of the cath team were not notified in a timely manner of the need for intervention. The patient deteriorated further, developing cardiopulmonary arrest (CPA) and subsequently experienced a fatal outcome which may have been partly attributed to lack of coordination among the care team.

The patient's minute-by-minute status information is accessible via the graphical user interface of the patient care surveillance system 10, which includes the patient's outcome. The status information can be viewed by members of hospital leadership, including the chief medical officer (CMO), the chief nursing officer (CNO), and the chief quality officer (CQO). This information may be used by the leadership to implement new procedures and policies to so that preventable adverse events are avoided. This could include items such as earlier activation of the RAT team and earlier transport/transfer of the patient to the appropriate unit especially for conditions where time-to-treatment is a significant predictor of patient outcomes. The facility may dedicate certain beds on a specific unit where patients who are determined to be at high risk by the predictive model for specific conditions, such as sepsis, cardiopulmonary arrest, and hypoglycemia, could be more closely monitored.

In another example, the same patient described above arrives at the emergency department in the same condition and with the same medical history. However unlike the prior example, the patient's medical information is immediately analyzed by the predictive model of the patient care surveillance system 10, which determines that the patient is at high risk for cardiopulmonary arrest. The admitting physician can automatically be notified of the high risk indication or the information can be accessed in system 10 by the medical director who immediately recommends to the attending physician that the patient be transferred to the ICU for close monitoring due to his CPA risk status.

As before, the patient's electrocardiogram (ECG) and cardiac enzyme test results become available and are stored for analysis and review via the graphical user interface of the patient care surveillance system 10. The rapid assessment team (RAT) is alerted of the occurrence of an acute heart attack via a page automatically transmitted by the system 10. The RAT is immediately mobilized, and they facilitate expedited transfer to the cath lab. The system 10 monitors to ensure all interventions are timely and properly administered. As a result, the patient receives appropriate intervention. The medical director alerts the attending physician to provide the patient with a mobile tablet to log any discomfort during the remainder of his stay in the ICU to engage the patient in managing his condition and proactively addressing any abnormalities to avoid a future adverse event.

The real-time data from the system 10 provides medical leadership the necessary information to make critical, time-sensitive, and evidence-based decisions to proactively avoid a likely adverse event. In this case, because of the patient's high risk for CPA, he is transferred to the ICU proactively where close monitoring and expedited treatment are possible. As such, the patient is better positioned to avoid the occurrence of the adverse event.

By analyzing real-time and historical patient data, the patient care surveillance system and method 10 is operable to provide disease identification, risk identification, and adverse event identification, so that the healthcare staff may proactively diagnose and treat the patients, and the patient's status may be continually anticipated, evaluated, and monitored. The system 10 helps to enforce time requirements for proscribed treatments and therapies, and automatically notifies the healthcare staff of status changes and/or impending treatment time window expirations. The patient data can be analyzed and evaluated to determine ways to improve the hospital's procedures and policies to provide better patient outcomes and efficient use of staff and resources.

The patient care surveillance system and method 10 are operable to generate various standard and custom reports. This output may be transmitted wirelessly or via LAN, WAN, the Internet (in the form of electronic fax, email, SMS, MMS, etc.), and delivered to healthcare facilities' electronic medical record stores, user electronic devices (e.g., pager, mobile telephone, tablet computer, mobile computer, laptop computer, desktop computer, and server), health information exchanges, and other data stores, databases, devices, and users.

The features of the present invention which are believed to be novel are set forth below with particularity in the appended claims. However, modifications, variations, and changes to the exemplary embodiments described above will be apparent to those skilled in the art, and the patient care surveillance system and method described herein thus encompasses such modifications, variations, and changes and are not limited to the specific embodiments described herein.

What is claimed is:

1. A patient care surveillance system, comprising:
a data store operable to receive and store clinical and non-clinical data associated with at least one patient;
a user interface configured to receive user input of current information related to the at least one patient;
a monitor configured to sense at least one parameter associated with the at least one patient and further configured to generate real-time patient monitor data;
a data analysis module configured to access the data store and analyze the clinical and non-clinical data, receive and analyze the current information and real-time patient monitor data, and identify at least one adverse event associated with the care of the at least one patient; and
a data presentation module operable to present information associated with the identified at least one adverse event to a healthcare professional.

2. The patient care surveillance system of claim 1, further comprising a data analysis module configured to access the data store and analyze the clinical and non-clinical data, receive and analyze the current information and real-time patient monitor data, and identify at least one disease associated with the at least one patient.

3. The patient care surveillance system of claim 1, further comprising a data analysis module configured to access the data store and analyze the clinical and non-clinical data, receive and analyze the current information and real-time patient monitor data, and identify at least one hospital readmission risk associated with the at least one patient.

4. The patient care surveillance system of claim 1, further comprising a data analysis module configured to access the data store and analyze the clinical and non-clinical data, receive and analyze the current information and real-time patient monitor data, and identify at least one recommended treatment option for the at least one patient.

5. The patient care surveillance system of claim 1, wherein the data analysis module comprises a natural language processing module.

6. The patient care surveillance system of claim 1, wherein the data analysis module comprises a data integration module configured to perform data extraction, cleansing, and manipulation.

7. The patient care surveillance system of claim 1, wherein the data analysis module comprises a predictive model.

8. The patient care surveillance system of claim 1, wherein the data analysis module comprises an artificial intelligence tuning module configured to fine tune the data analysis based on actual observed outcomes compared to predicted outcomes to provide more accurate results.

9. The patient care surveillance system of claim 1, wherein the clinical and non-clinical data are selected from the group consisting of: past medical history, age, weight, height, race, gender, marital status, education, address, housing status, allergy and adverse medical reactions, family medical information, prior surgical information, emergency room records, medication administration records, culture results, clinical notes and records, gynecological and obstetric information, mental status examination, vaccination records, radiological imaging exams, invasive visualization procedures, psychiatric treatment information, prior histological specimens, laboratory results, genetic information, socio-economic status, type and nature of employment, job history, lifestyle, hospital utilization patterns, addictive substance use, frequency of physician or health system contact, location and frequency of habitation changes, census and demographic data, neighborhood environments, diet, proximity and number of family or care-giving assistants, travel history, social media data, social workers' notes, pharmaceutical and supplement intake information, focused genotype testing, medical insurance information, exercise information, occupational chemical exposure records, predictive screening health questionnaires, personality tests, census and demographic data, neighborhood environment data, and participation in food, housing, and utilities assistance registries.

10. The patient care surveillance system of claim 1, wherein the user interface is configured to receive user input of a patient's symptoms.

11. The patient care surveillance system of claim 1, wherein the monitor comprises a vital signs monitor configured to continually measure the at least one patient's vital signs and transmit the vital signs data for analysis by the data analysis module.

12. The patient care surveillance system of claim 1, wherein the monitor comprises at least one presence sensor configured to sense and monitor the presence of the at least one patient.

13. The patient care surveillance system of claim 1, wherein the monitor comprises a plurality of RFID sensors configured to sense the presence of an RFID tag on the at least one patient.

14. The patient care surveillance system of claim 1, wherein the monitor comprises a subcutaneous glucose sensor configured to measure a blood glucose level of the at least one patient.

15. The patient care surveillance system of claim 1, wherein the monitor comprises at least one video camera configured to capture moving images of the at least one patient.

16. The patient care surveillance system of claim 1, wherein the data presentation module is configured to receive user input of parameters specifying an adverse event type, a time window, and unit of interest.

17. The patient care surveillance system of claim 1, wherein the data presentation module is configured to present a graphical representation of relevant data.

18. The patient care surveillance system of claim 1, wherein the data presentation module is configured to present a list view communicating one of: a list of patients with impending failures on any aspect of the metric under consideration (risk view), and a list of patients who actually failed on any aspect of the metric under consideration (event view).

19. The patient care surveillance system of claim 1, wherein the data presentation module is configured to present a pareto view communicating at least one of the total number and percentage of actual failures on any aspect of the metric under consideration (event view), and the total number of patients who actually failed on any aspect of the metric under consideration (pareto list view).

20. The patient care surveillance system of claim 1, wherein the data presentation module is configured to present a failure view communicating at least one of the metric failure(s) encountered by each patient.

21. The patient care surveillance system of claim 1, wherein the data presentation module is configured to present a tile view communicating at least one of the total number of patients with an impending failure for the specific adverse event under consideration (risk view), and the total number of patients who actually failed for each specific adverse event under consideration (event view).

22. The patient care surveillance system of claim 1, wherein the data store comprises a plurality of databases.

23. The patient care surveillance system of claim 1, wherein the data analysis module is configured to issue a notification, and the data presentation module is configured to transmit the notification to personnel relevant to the care of the at least one patient.

24. The patient care surveillance system of claim 1, wherein the data analysis module is configured to issue a notification, and the data presentation module is configured to transmit the notification in the form of at least one of a page, a text message, a voice message, an email message, a telephone call, and a multimedia message to personnel relevant to the care of the at least one patient.

25. The patient care surveillance system of claim 1, wherein the data analysis module is configured to issue a notification in response to the at least one patient's status being inconsistent with an expected status, and the data presentation module is configured to transmit the notification to personnel relevant to the care of the at least one patient.

26. The patient care surveillance system of claim 1, wherein the data analysis module is configured to issue a notification in response to an ordered activity associated with the at least one patient being incomplete within a required time period, and the data presentation module is configured to transmit the notification to personnel relevant to the care of the at least one patient.

27. The patient care surveillance system of claim 1, wherein the data analysis module is configured to issue a notification in response to a monitored location of the at least one patient being inconsistent with an ordered treatment for the patient, and the data presentation module is configured to transmit the notification to personnel relevant to the care of the at least one patient.

28. A patient care surveillance method, comprising:
accessing stored clinical and non-clinical data associated with at least one patient;
receiving user input of current information related to the at least one patient;
sensing at least one parameter associated with the at least one patient, and further generating real-time patient monitor data;
analyzing the clinical and non-clinical data, receiving and analyzing the current information and real-time patient monitor data, and identifying at least one adverse event associated with the care of the at least one patient; and
presenting information associated with identification of at least one adverse event to a healthcare professional.

29. The patient care surveillance method of claim 28, further comprising accessing the data store and analyzing the clinical and non-clinical data, receiving and analyzing the current information and real-time patient monitor data, and identifying at least one disease associated with at least one patient.

30. The patient care surveillance method of claim 28, further comprising accessing the data store and analyzing the clinical and non-clinical data, receiving and analyzing the current information and real-time patient monitor data, and identifying at least one hospital readmission risk associated with the at least one patient.

31. The patient care surveillance method of claim 28, further comprising accessing the data store and analyzing the clinical and non-clinical data, receiving and analyzing the current information and real-time patient monitor data, and identifying at least one recommended treatment option for the at least one patient.

32. The patient care surveillance method of claim 28, further comprising accessing the data store and analyzing the clinical and non-clinical data, receiving and analyzing the current information and real-time patient monitor data, and identifying at least one recommended course of action for the at least one patient.

33. The patient care surveillance method of claim 28, wherein analyzing the data comprises performing natural language processing, data extraction, data cleansing, and data manipulation.

34. The patient care surveillance method of claim 28, wherein analyzing the data comprises fine tuning the data analysis based on actual observed outcomes compared to predicted outcomes to provide more accurate results.

35. The patient care surveillance method of claim 28, wherein receiving and analyzing the clinical and non-clinical data comprises receiving and analyzing data selected from the group consisting of: past medical history, age, weight, height, race, gender, marital status, education, address, housing status, allergy and adverse medical reactions, family medical information, prior surgical information, emergency room records, medication administration records, culture results, clinical notes and records, gynecological and obstetric information, mental status examination, vaccination records, radiological imaging exams, invasive visualization procedures, psychiatric treatment information, prior histological specimens, laboratory results, genetic information, socio-economic status, type and nature of employment, job history, lifestyle, hospital utilization patterns, addictive substance use, frequency of physician or health system contact, location and frequency of habitation changes, census and demographic data, neighborhood environments, diet, proximity and number of family or care-giving assistants, travel history, social media data, social workers' notes, pharmaceutical and supplement intake information, focused genotype testing, medical insurance information, exercise information, occupational chemical exposure records, predictive screening health questionnaires, personality tests, census and demographic data, neighborhood environment data, and participation in food, housing, and utilities assistance registries.

36. The patient care surveillance method of claim 28, wherein receiving user input comprises receiving user input of patient's symptoms.

37. The patient care surveillance method of claim 28, wherein sensing at least one parameter comprises continually measuring the at least one patient's vital signs and transmitting the vital signs data for analysis.

38. The patient care surveillance method of claim 28, wherein sensing at least one parameter comprises sensing and monitoring the presence of the at least one patient.

39. The patient care surveillance method of claim 28, wherein sensing at least one parameter comprises sensing the presence of an RFID tag on the at least one patient.

40. The patient care surveillance method of claim 28, wherein sensing at least one parameter comprises measuring a blood glucose level of at least one patient.

41. The patient care surveillance method of claim 28, wherein sensing at least one parameter comprises capturing still and moving images of at least one patient.

42. The patient care surveillance method of claim 28, wherein presenting information comprises receiving user input of parameters specifying an adverse event type, a time window, and unit of interest.

43. The patient care surveillance method of claim 28, wherein presenting information comprises presenting a graphical representation of relevant data.

44. The patient care surveillance system of claim 28, wherein the data presentation module is configured to present a list view communicating one of: a list of patients with impending failures on any aspect of the metric under consideration (risk view), and a list of patients who actually failed on any aspect of the metric under consideration (event view).

45. The patient care surveillance system of claim 28, wherein the data presentation module is configured to present a pareto view communicating at least one of the total number and percentage of actual failures on any aspect of the metric under consideration (event view), and the total number of patients who actually failed on any aspect of the metric under consideration (pareto list view).

46. The patient care surveillance system of claim 28, wherein the data presentation module is configured to present a failure view communicating at least one of the metric failure(s) encountered by each patient.

47. The patient care surveillance system of claim 28, wherein the data presentation module is configured to present a tile view communicating at least one of the total number of patients with an impending failure for the specific adverse event under consideration (risk view), and the total number of patients who actually failed for each specific adverse event under consideration (event view).

48. The patient care surveillance method of claim 28, further comprising issuing a notification, and transmitting the notification to personnel relevant to the care of the at least one patient.

49. The patient care surveillance method of claim 28, further comprising issuing a notification, and transmitting the notification in the form of at least a page, a text message, a voice message, an email message, a telephone call, or a multimedia message to personnel relevant to the care of the at least one patient.

50. The patient care surveillance method of claim 28, further comprising issuing a notification in response to at least one patient's status is inconsistent with an expected status, and transmitting the notification to personnel relevant to the care of the at least one patient.

51. The patient care surveillance method of claim 28, further comprising issuing a notification in response to an ordered activity associated with the at least one patient being incomplete within a required time period, and transmitting the notification to personnel relevant to the care of the at least one patient.

52. The patient care surveillance method of claim 28, further comprising issuing a notification in response to a monitored location of the at least one patient being inconsistent with an ordered treatment for the patient, and transmitting the notification to personnel relevant to the care of the at least one patient.

53. The patient care surveillance method of claim 28, wherein presenting information comprises presenting contextual information associated with the data.

54. A computer-readable medium having encoded thereon a process for patient care surveillance, the process comprising:
accessing stored clinical and non-clinical data associated with the at least one patient;
receiving user input of current information related to the at least one patient;
sensing at least one parameter associated with at least one patient, and further generating real-time patient monitor data;
analyzing the clinical and non-clinical data, receiving and analyzing the current information and real-time patient monitor data, and identifying at least one course of action associated with the care of the at least one patient; and
presenting information associated with at least one course of action to a healthcare professional.
